(19) United States
(12) Patent Application Publication
(10) Pub. No.: US 2018/0371509 A1
(43) Pub. Date: Dec. 27, 2018

(54) PHA-PRODUCING MICROORGANISM HAVING SUCROSE ASSIMILABILITY, AND METHOD FOR PRODUCING PHA USING SAID MICROORGANISM

(71) Applicant: KANEKA CORPORATION, Osaka-shi, Osaka (JP)

(72) Inventors: Hisashi ARIKAWA, Takasago-shi (JP); Tetsuya FUJIKI, Osaka-shi (JP)

(73) Assignee: KANEKA CORPORATION, Osaka-shi, Osaka (JP)

(21) Appl. No.: 16/061,923

(22) PCT Filed: Dec. 14, 2016
(86) PCT No.: PCT/JP2016/087292

§ 371 Date: Jun. 13, 2018

(30) Foreign Application Priority Data
Dec. 16, 2015 (JP) 2015-245214

(51) Int. Cl. C12P 7/62; C08G 63/06; C12N 9/10; C12N 15/52; C12N 1/20

(52) U.S. Cl. CPC C12P 7/625; C08G 63/06; C12N 9/1062; C12Y 204/01013; C12N 15/52; C12N 1/20; C12N 9/1025

(57) ABSTRACT

An object of the present invention is to provide a PHA-producing microorganism which can assimilate sucrose, and a method for producing a PHA by culturing this microorganism, using sucrose as a carbon source. A PHA-producing microorganism, comprising a PHA synthase gene, and heterogeneous-organism-derived genes in the following items (1) and (2): (1) a sucrose hydrolase gene encoding an amino acid sequence described in SEQ ID NO: 1, or a gene encoding a polypeptide which has a sequence homology of 90% or more to the amino acid sequence and which has sucrose hydrolase activity; and (2) a sucrose permease gene encoding an amino acid sequence described in SEQ ID NO: 2, or a gene encoding a polypeptide which has a sequence homology of 90% or more to the amino acid sequence and which has sucrose permease activity. The invention is also a method for producing a PHA, including the step of culturing this microorganism in a medium including sucrose as a carbon source.

### TECHNICAL FIELD

The present invention relates to a PHA-producing microorganism having sucrose assimilability, and a method for producing a PHA using the microorganism.

### BACKGROUND ART

A polyhydroxyalkanoic acid (hereinafter referred to also as a PHA) is a polyester type organic polymer produced by many microorganism species. The PHA is a thermoplastic polymer having biodegradability, and can be produced using a renewable source as a raw material. From these matters, trails have been made for producing the PHA industrially as an environment harmony type material or biocompatible material, and making use of the PHA for various industries.

A general name of the species of monomer units constituting this PHA is a hydroxyalkanoic acid. Specific examples of the hydroxyalkanoic acid include 3-hydroxybutyric acid (hereinafter referred to also as 3HB), 3-hydroxyvaleric acid (hereinafter referred to also as 3HV), 3-hydroxyhexanoic acid (hereinafter referred to also as 3HH), 3-hydroxyoctanoic acid, 3-hydroxyalkanoic acids each having a longer alkyl chain, and 4-hydroxybutyric acid. One or more of these acids are homo-polymerized or copolymerized to produce a PHA, which is a polymeric molecule.

A specific example of the PHA is poly-3-hydroxybutyric acid (hereinafter referred to also as P(3HB)), which is a homopolymer of 3HB. Other specific examples thereof include P(3HB-co-3HV), which is a copolymer made from 3HB and 3HV, and P(3HB-co-3HH) (hereinafter referred to also as PHBH), which is a copolymer made from 3HB and 3HH, and P(3HB-co-4HB), which is a copolymer made from 3HB and 4HB. Out of these examples, particularly, PHBH can be caused to have broad physical properties applicable in a scope from hard polymers to soft polymers by varying the 3HH composition ratio therein. Thus, about PHBH, applications thereof to broad fields are expectable, examples of articles in the fields including articles required to be hard such as a television case using PHBH having a low 3HH composition ratio, and articles required to be flexible such as a film using PHBH having a high 3HH composition ratio.

In a fermentation production of bulk chemicals such as a PHA, the proportion of carbon source costs in costs for the production is large. It is therefore important to use an inexpensive carbon source effectively for fermentation.

In the production of chemical substances by microorganism fermentation, glucose is used as a main carbon source in many cases. However, glucose is a relatively expensive carbon source. Accordingly, the use of glucose as a main carbon source may make production costs of the resultant higher than those based on a chemical synthesis method using crude petroleum as a main raw substance. Thus, it is difficult to commercialize the former from the viewpoint of price competitive power.

In the meantime, as a saccharide material more inexpensive than glucose, sucrose is known. Sucrose is a disaccharide made from glucose and fructose, is produced by all plants having photosynthesis capability, and is a carbon source very abundant in the nature. Furthermore, sucrose is a main component of molasses, and is an appealing carbon source also in the point that the molasses is a renewable source which is not competitive with food.

According to Patent Literature 1, the mechanism that microorganisms assimilate sucrose is roughly classified into the following two: sucrose PTS (phosphoenolpyruvate: carbohydrate phosphotransferase system) and sucrose non-PTS. When the assimilation goes through the sucrose non-PTS, a microorganism takes in sucrose as it is, and subsequently decomposes sucrose into glucose and fructose. In the meantime, when the assimilation goes through the sucrose PTS, a microorganism phosphorylates sucrose at the time of uptaking it, so that sucrose is converted to sucrose-6-phosphate. The microorganism then decomposes, in cells thereof, sucrose 6-phosphate into glucose 6-phosphate and fructose. Alternatively, outside of the cells of the microorganism, sucrose is decomposed, and the resultant materials glucose and fructose may be assimilated.

However, not all microorganisms can assimilate sucrose. For example, a Cupriavidus necator H16 strain, which is known as a PHA-producing microorganism, can assimilate fructose, but cannot assimilate glucose nor sucrose.

So far, several researches have been made about which sucrose assimilability is given to a microorganism having no sucrose assimilability by a genetic engineering technique. For example, Non Patent Literature 1 discloses that sucrose-assimilation-related genes (a sucrose permease gene and a sucrose hydrolase gene) derived from an Escherichia coli W strain are introduced to an Escherichia coli K-12 strain which does not originally have sucrose assimilability, so that the K-12 strain can come to assimilate sucrose. In this case, an organism from which the genes are derived, and the host into which the genes are to be introduced are the same as each other in species. Thus, the probability would be high that the genes introduced by the gene recombination function satisfactorily. Patent Literature 2 discloses an example in which sucrose assimilability is given to bacteria in the genus Escherichia, which have no sucrose assimilability, by introducing a group of sucrose PTS genes thereinto. Furthermore, Patent Literature 1 discloses an example in which sucrose assimilability is given to a host by introducing, thereinto, a sucrose phosphotransferase gene and a sucrose hydrolase gene.

### CITATION LIST

### Patent Literatures

PTL 1: JP 2011-505869A

PTL 2: JP 2001-346578 A

### Non Patent Literature

NPTL 1: Appl. Environ. Microbiol., vol. 79, 478 (2013)

### SUMMARY OF INVENTION

### Technical Problem

An object of the present invention is to provide a PHA-producing microorganism which can assimilate sucrose, and a method for producing a PHA by culturing this microorganism using sucrose as a carbon source.

### Solution to Problem

In order to solve the above-mentioned problems, the inventors have made eager researches to find out that by introducing, into a microorganism having a capability of producing a PHA, both of a heterogeneous-organism-derived gene encoding a sucrose hydrolase (CscA) and a heterogeneous-organism-derived gene encoding a sucrose permease (CscB), a sucrose decomposing capability and a capability of taking sucrose into cells are given to the microorganism, so that PHA production can be attained using sucrose as a carbon source. In this way, the invention has been accomplished.

Thus, the present invention relates to a PHA-producing microorganism including a PHA synthase gene, and heterogeneous-organism-derived genes in the following items (1) and (2):

(1) a sucrose hydrolase gene encoding an amino acid sequence described in SEQ ID NO: 1, or a gene encoding a polypeptide which has a sequence homology of 90% or more to the amino acid sequence and which has sucrose hydrolase activity, and
(2) a sucrose permease gene encoding an amino acid sequence described in SEQ ID NO: 2, or a gene encoding a polypeptide which has a sequence homology of 90% or more to the amino acid sequence and which has sucrose permease activity.

The microorganism is preferably a transformant in which a microorganism belonging to a genus Cupriavidus is used as a host. The microorganism belonging to the genus Cupriavidus is more preferably Cupriavidus necator. Preferably, glucose assimilability is given to the microorganism or glucose assimilability is enhanced in the microorganism. The PHA synthase gene is preferably a PHA synthase gene capable of synthesizing P(3HB-co-3HH). The microorganism preferably further includes a crotonyl-CoA reductase gene, and an ethylmalonyl-CoA decarboxylase gene. Preferably, the microorganism is a microorganism in which an acetoacetyl CoA reductase gene is deleted, or an expression level thereof is restrained.

The present invention also relates to a method for producing a PHA including the step of culturing the above-defined microorganism in a medium containing sucrose as a carbon source, and is preferably the producing method in which the PHA is P(3HB-co-3HH).

### Advantageous Effects of Invention

The present invention makes it possible to provide a PHA-producing microorganism which can assimilate sucrose. The invention also makes it possible to produce a PHA through fermentation by culturing the microorganism using sucrose as a carbon source.

### BRIEF DESCRIPTION OF DRAWINGS

FIG. 1 is a graph about Comparative Example 1 that shows growth of a KNK005ΔphaZ1,2,6 strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 2 is a graph about Comparative Example 2 that shows growth of a KNK005ΔphaZ1,2,6/nagEG793C,dR strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 3 is a graph about Comparative Example 3 that shows growth of a pCUP2-lacUV5-cscA in KNK005ΔphaZ1,2,6 strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 4 is a graph about Comparative Example 4 that shows growth of a pCUP2-lacUV5-cscB in KNK005ΔphaZ1,2,6 strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 5 is a graph about Example 1 that shows growth of a pCUP2-lacUV5-cscAB in KNK005ΔphaZ1,2,6 strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 6 is a graph about Example 2 that shows growth of a pCUP2-lacUV5-cscAB in KNK005ΔphaZ1,2,6/nagEG793C,dR strain in a sucrose-containing medium and a change in the concentration of each saccharide in the medium (solid line containing ●: OD₆₀₀; solid line containing Δ: sucrose concentration; solid line containing ▪: glucose concentration; and solid line containing x: fructose concentration).

FIG. 7 is a graph about Example 2 that shows growth of a pCUP2-lacUV5-cscAB in KNK005ΔphaZ1,2,6/nagEG793C,dR strain in a sucrose-containing medium, a glucose-containing medium or a fructose-containing medium, the weight of dried bacterial cells therein, and a change in the amount of a produced PHA therein (solid line containing change Δ: in the sucrose-containing medium; solid line containing ▪: change in the glucose-containing medium; and solid line containing x: a change in the fructose-containing medium).

### DESCRIPTION OF EMBODIMENTS

Hereinafter, the present invention will be described in more detail.

(1) PHA-Producing Microorganism to which Sucrose Assimilability is Given or in which Sucrose Assimilability is Enhanced

The present invention includes introducing, into a microorganism having a PHA synthase gene, both of a sucrose hydrolase gene and a sucrose permease gene, these genes being each derived from an organism different in species from the microorganism, thereby providing a microorganism to which sucrose assimilability is given or in which sucrose assimilability is enhanced so that a PHA is produced using sucrose as a carbon source.

The wording “sucrose hydrolase gene” used in the DESCRIPTION is a gene encoding a sucrose hydrolase (CscA), which is an enzyme which hydrolyzes sucrose to produce glucose and fructose. The wording “sucrose permease gene” used in the DESCRIPTION is a gene encoding a sucrose permease (CscB), which has a function of taking sucrose into cells.

In the present invention, the microorganism which is an original strain (host) into which the sucrose hydrolase gene and the sucrose permease gene are introduced is not particularly limited as long as the microorganism is a microorganism which has a PHA synthase gene and can produce a PHA. The microorganism is preferably a PHA-producing microorganism which does not originally have sucrose assimilability, or which is low in sucrose assimilability. Such a PHA-producing microorganism may be, as well as a wild strain which originally has a PHA synthase gene, a variant obtained by subjecting such a wild strain artificially to spontaneous mutation treatment, or a recombinant bacterial strain in which a gene engineering technique is used to introduce a PHA synthase foreign gene into a microorganism having no PHA synthase gene originally.

Specific examples of the microorganism include bacteria, yeasts, and filamentous fungi. The microorganism is preferably a bacterium. Preferred examples of the bacterium include bacterial belonging to the genus Ralstonia, the genus Cupriavidus, the genus Wautersia, the genus Aeromonas, the genus Escherichia, the genus Alcaligenes, and the genus Pseudomonas. From safety and the producing performance, more preferred are bacteria belonging to the genus Ralstonia, the genus Cupriavidus, the genus Aeromonas, and the genus Wautersia, even more preferred are bacteria belonging to the genus Cupriavidus or the genus Aeromonas, and even more preferred are bacteria belonging to the genus Cupriavidus. Particularly preferred is Cupriavidus necator.

When the microorganism having a PHA synthase gene is a recombinant bacterial strain into which a gene engineering technique is used to introduce a PHA synthase foreign gene, examples of the PHA synthase foreign gene include a PHA synthase gene encoding an amino acid sequence described in SEQ ID NO: 3, this gene being possessed by a Cupriavidus necator H16 strain (C. necator H16) strain; a PHA synthase gene encoding a polypeptide which has a sequence homology of 85% or more to the amino acid sequence and which has PHA synthesis activity; a PHA synthase gene which Aeromonas caviae has; and a PHA synthase gene encoding a polypeptide which has a sequence homology of 85% or more to the amino acid sequence and which has PHA synthesis activity. However, the PHA synthase foreign gene is not limited to these examples. Thus, other PHA synthase genes are also favorably usable. The sequence homology is preferably 90% or more, more preferably 95% or more, and particularly preferably 99% or more. Out of these examples, preferred is a PHA synthase gene which can synthesize PHBH as a PHA, and more preferred is, for example, a PHA synthase gene encoding a PHA synthase having an amino acid sequence described in SEQ ID NO: 4.

In the present invention, the microorganism which is an original strain into which the sucrose hydrolase gene and the sucrose permease gene are introduced is preferably a microorganism having a PHA synthase gene which can synthesize PHBH. A specific example thereof is most preferably a variant in which a PHA synthase gene derived from Aeromonas caviae is introduced to C. necator H16.

In the present invention, the following are introduced to the above-mentioned microorganism: a sucrose hydrolase gene and a sucrose permease gene that are each derived from an organism different in species from this microorganism. The sucrose hydrolase gene is not particularly limited as long as the gene is a gene encoding an amino acid sequence described in SEQ ID NO: 1, this gene being derived from Escherichia coli, or a gene encoding a polypeptide which has a sequence homology of 90% or more to the amino acid sequence and which has sucrose hydrolase activity. An example thereof is a gene having a base sequence described in SEQ ID NO: 5. The sucrose permease gene is not particularly limited as long as the gene is a gene encoding an amino acid sequence described in SEQ ID NO: 2, this gene being derived from Escherichia coli, or a gene encoding a polypeptide which has a sequence homology of 90% or more to the amino acid sequence and which has sucrose permease activity. An example thereof is a gene having a base sequence described in SEQ ID NO: 6. The above-mentioned sequence homologies are each preferably 95% or more, more preferably 97% or more, and particularly preferably 99% or more.

In the PHA-producing microorganism of the present invention, the PHA synthase gene, the sucrose hydrolase gene, and the sucrose permease gene may each present on a DNA in a chromosome, a plasmid, a megaplasmid, or any other that the microorganism which becomes a host has; or on a DNA artificially introduced, for example on a plasmid vector, an artificial chromosome or some other. However, from the viewpoint of the retention of the introduced DNA, the genes are each preferably present on a chromosome or a megaplasmid which the microorganism has, and are each more preferably present on a chromosome which the microorganism has.

A method for applying a site-specific substitution or insertion of any DNA onto a DNA which a microorganism has is well known, and the method is usable when the microorganism of the present invention is produced. The method is not particularly limited, and typical examples thereof include a method using a mechanism of transposon and homologous recombination (Ohman et al., J. Bacteriol., vol. 162, p. 1068 (1985)); a method using, as a principle, site-specific introduction caused by a homologous recombination mechanism, and dropout based on homologous recombination at the second stage (Noti et al., Methods Enzymol., vol. 154, p. 197 (1987)); and a method of causing a sacB gene derived from Bacillus subtilis to coexist, and then isolating, as a sucrose-added-medium resistant strain, a microorganism strain in which a gene is dropped out by homologous recombination at the second stage (Schweizer, Mol. Microbiol., vol. 6, p. 1195 (1992), and Lenz et al., J. Bacteriol., vol. 176, p. 4385 (1994)). The method for introducing a vector into a cell is not particularly limited, either. Examples thereof include a calcium chloride method, an electroporation method, a polyethylene glycol method, and a spheroplast method.

About gene cloning and gene recombination techniques, techniques described in the following are usable: for example, Sambrook, J. et al., Molecular Cloning, A Laboratory Manual, Cold Spring Harbor Laboratory Press (1989 or 2001).

A promoter for expressing each of the sucrose hydrolase gene and the sucrose permease gene is not particularly limited. For example, the following is usable: a promoter of a phaC1 gene or a promoter of a phaP1 gene of Cupriavidus necator; or a lac promoter, a lacUV5 promoter, a trc promoter, a tic promoter or a tac promoter derived from Escherichia coli. For each of the genes, the same promoters may be used, or different promoters may be used. It is particularly preferred to use, for both the genes, a lacUV5 promoter described in SEQ ID NO: 7.

When the microorganism to which sucrose assimilability is given or in which sucrose assimilability is enhanced has a low glucose assimilability or no glucose assimilability, it is preferred to give glucose assimilability to the microorganism or enhance the glucose assimilability thereof by, for example, a method of gene mutation, gene disruption, the enhancement of gene expression, or foreign gene introduction. In this way, the sucrose assimilability of the microorganism is further enhanced, and in the case of using sucrose as the carbon source, the amount of PHA production can be improved. For example, a C. necator H16 strain has no gene for taking glucose thereinto. Thus, the strain cannot assimilate glucose. The method for giving glucose assimilability to the C. necator H16 strain is not particularly limited. An example thereof is a method of substituting G which is the 793^{th} base in nagF, which is a gene for taking in N-acetyl glucosamine, with C, and further disrupting nagR, which is a gene encoding a transcriptional regulator, to give glucose assimilability thereto (Journal of Bioscience and Bioengineering, vol. 113, 63(2012)). Moreover, another example thereof is a method of introducing a foreign gene encoding a glucose transporter into the strain to give glucose assimilability thereto (JP 2009-225662). Furthermore, a method of introducing a glucose-phosphorylation enzyme gene into the strain may be useful.

According to research in the prior art, it is known that PHBH can be produced using fructose as a carbon source by introducing a crotonyl-CoA reductase gene (ccr) and an ethylmalonyl-CoA decarboxylase gene (emd) into Cupriavidus necator into which a PHA synthase gene which can take in a 3HH monomer is introduced, or by introducing a gene (phaJ) encoding an (R)-specific enoyl-CoA hydratase further thereinto (Metabolic Engineering, vol. 27, 38(2015)). It is allowable to introduce, into the PHA-producing microorganism of the present invention, ccr and/or emd in addition to the sucrose hydrolase gene and the sucrose pelt lease gene. When carbohydrate is used as a carbon source, the introduction of ccr and/or emd makes it possible to enhance a synthesis route of (R)-3-hydroxyacyl-CoA having 6 carbon atoms, or make the route efficient. Thus, in PHBH produced, the 3HH composition ratio can be improved.

The crotonyl-CoA reductase used in the present invention is an enzyme for reducing crotonyl-CoA having 4 carbon atoms, which is an intermediate in an aliphatic acid β-oxidizing route, to produce butyryl-CoA. Butyryl-CoA is condensed with another molecule acetyl-CoA by effect of β-ketothiolase (BktB), and is further converted to supply (R)-3HHx-CoA having 6 carbon atoms. This substance is copolymerized with (R)-3HB-CoA by aid of a polyester polymerizing enzyme showing a broad substrate specificity. The gene ccr usable in the present invention is not particularly limited as long as the reductase yielded after the gene is translated has the activity of the above-mentioned crotonyl-CoA reductase. Examples thereof include a gene encoding a crotonyl-CoA reductase derived from S. cinnamonensis (Gene Bank Accession No. AF178673), and a gene encoding a crotonyl-CoA reductase derived from a methanol assimilating bacterium M. extorquen (NCBI-Gene ID: 7990208). Preferred examples thereof include a gene encoding a crotonyl-CoA reductase having an amino acid sequence described in SEQ ID NO: 40, and a gene encoding a polypeptide which has a sequence homology of 90% or more to the amino acid sequence and which has crotonyl-CoA reductase activity.

The ethylmalonyl-CoA decarboxylase used in the present invention is an enzyme which catalyzes a decarboxylating reaction of ethylmalonyl-CoA, which is generated by side reaction by aid of, e.g., the crotonyl-CoA reductase or propionyl-CoA carboxylase, to butyryl-CoA. The derivation of the ethylmalonyl-CoA decarboxylase is not particularly limited as long as this enzyme has this activity. The enzyme is, for example, an ethylmalonyl-CoA decarboxylase derived from a mouse and having an amino acid sequence described in SEQ ID NO: 41. A gene base sequence which encodes this amino acid sequence and is usable in Cupriavidus necator is, for example, a base sequence described in SEQ ID NO: 42. However, the gene base sequence is not limited to this base sequence.

About the PHBH-producing microorganism according to the present invention, it is allowable to introduce, thereinto, not only the above-mentioned genes ccr and emd but also the above-mentioned gene (phaJ) encoding an (R)-specific enoyl-CoA hydratase. This introduction makes it possible to enhance biosynthesis power of (R)-3HHx-CoA. The (R)-specific enoyl-CoA hydratase used in the present invention means an enzyme which converts 2-enoyl-CoA which is an aliphatic acid β-oxidizing type intermediate, into (R)-3-hydroxyacyl-CoA which is a PHA monomer. As long as phaJ has this activity, the derivation of phaJ is not particularly limited. The derivation is preferably derived from Cupriavidus necator or Aeromonas caviae. The phaJ used in the present invention is, for example, phaJ4a (H16 A1070, NCBI-Gene ID: 4248689), or phaJ4b (H16 B0397, NCBI-Gene ID: 4454986) derived from Cupriavidus necator; or phaJ (Gene Bank Accession No. BAA21816) derived from Aeromonas caviae.

About the PHBH-producing microorganism of the present invention, PHBH higher in 3HH composition ratio can be produced by not only the introduction of the genes ccr and emd but also deletion of a gene encoding an acetoacetyl-CoA reductase therefrom or the restraint of the expression thereof. The gene encoding the acetoacetyl-CoA reductase which should be deleted or restrained from being expressed, is sufficient to be a gene encoding an enzyme having a catalytic function of producing (R)-3HB-CoA, using acetoacetyl-CoA as a substrate. The gene is not particularly limited, and examples thereof include phaB1, and phaB3 (NCBI-Gene ID: 4249784), and NCBI-Gene ID: 4250155).

In the DESCRIPTION, the deletion denotes that gene manipulation or mutation causes a target gene to be partially or wholly not to be present, or the addition or the substitution of a base sequence causes a termination codon to make its appearance or causes a change or some other in an amino acid sequence to be encoded, so that the activity of a protein encoded by this gene is partially or wholly deleted. The method for restraining the gene expression is, for example, a method of modifying a base sequence in a promoter region at the upstream side of the gene, or in a ribosome-binding-sequence. However, the method is not limited to this method.

In the PHA-producing microorganism of the present invention, ccr, emd, and phaJ may each be present on a DNA of a chromosome, a plasmid or a megaplasmid that a microorganism, which is a host, has or may be present on a DNA introduced artificially into the microorganism, for example, on a plasmid vector or an artificial chromosome. However, from the viewpoint of the retention of the introduced DNA, the genes are each preferably present on a chromosome or a megaplasmid which the microorganism has, and are each more preferably present on a chromosome which the microorganism has. When the microorganism, which is a host, originally has these genes, the expression level of the genes may be increased, for example, by the substitution, deletion or addition of a base sequence at the upstream side of a gene which the microorganism originally has.

The promoter for expressing each of the genes ccr, emd, and phaJ is not particularly limited. For example, the following is usable: a promoter of a phaC1 gene or a promoter of a phaP1 gene of Cupriavidus necator; or a lac promoter, a lacUV5 promoter, a trc promoter, a tic promoter or a tac promoter derived from Escherichia coli. For each of the genes, the same promoters may be used, or different promoters may be used. For each of the genes, the trc promoter is in particular preferably used.

(2) Method for Producing PHA

The microorganism of the present invention is cultured in a medium containing sucrose as a carbon source, thereby making it possible to produce a PHA. By collecting the resultant PHA, the production of the PHA is attained.

In the PHA production according to the present invention, the microorganism is preferably cultured in a medium containing a carbon source, and nutrition sources other than the carbon source, for example, a nitrogen source, inorganic salts, and other organic nutrition sources.

The carbon source is sufficient to contain sucrose. An example of the sucrose-containing carbon source is syrup or molasses containing sucrose plentifully. As long as the medium contains sucrose, the medium may contain any other carbon source. Sucrose and any other carbon source may be used in combination. As the other carbon source, any carbon source is usable as long as the microorganism of the present invention can assimilate. Preferred examples thereof include saccharides such as glucose and fructose; oils and fats such as palm oil, palm kernel oil, corn oil, coconut oil, olive oil, soybean oil, linseed oil, and Jatropha oil, and fractionized oils thereof; and aliphatic acids such as lauric acid, oleic acid, stearic acid, palmitic acid, and myristic acid, and derivatives thereof.

Examples of the nitrogen source include ammonia; ammonium slats such as ammonium chloride, ammonium sulfate, and ammonium phosphate; and peptone, meat extracts, and yeast extracts. Examples of the inorganic salts include potassium dihydrogenphosphate, sodium dihydrogenphosphate, magnesium phosphate, magnesium sulfate, and sodium chloride. Examples of the other organic nutrition sources include amino acids such as glycine, alanine, serine, threonine, and proline; and vitamins such as vitamin B1, vitamin B12, and vitamin C.

When the microorganism of the present invention is cultured, the culturing temperature, the culturing period, the pH in the culturing, the medium, and other conditions may be conditions used ordinarily used to culture bacteria in the genus Ralstonia, the genus Cupriavidus, the genus Wautersia, the genus Aeromonas, the genus Escherichia, the genus Alcaligenes, the genus Pseudomonas, and others.

The species of a PHA produced in the present invention is not particularly limited as long as the produced PHA is a PHA which can be produced by a microorganism, and is preferably a PHA yielded by polymerizing one or more monomers selected from hydroxyalkanoic acids each having 4 to 16 carbon atoms. Examples thereof include P(3HB) which is a homopolymer of 3HB, P(3HB-co-3HV) which is a copolymer made from 3HB and 3HV, PHBH which is a copolymer made from 3HB and 3HH, and P(3HB-co-4HB) which is a copolymer made from 3HB and 4HB. However, the produced PHA is not limited to these polymers. Out of these examples, PHBH is preferred because of a wide application scope of this compound as a polymer. The species of the produced PHA is appropriately selectable in accordance with the species of a PHA synthase gene which the used microorganism has or which is separately introduced, the species of genes of a metabolic system related to the synthesis, the culturing conditions, and others, correspondingly to a purpose of the PHA.

After the microorganism is cultured in the present invention, the collection of the PHA from the bacterial cells is not particularly limited, and can be attained by, for example, the following method. After the end of the culturing, for example, a centrifugal separator is used to separate the bacterial cells from the cultured liquid. The bacterial cells are washed with, for example, distillated water or methanol, and dried. From the dried bacterial cells, an organic solvent such as chloroform is used to extract the PHA. Form this PHA-containing solution in the organic solvent, bacterial cell components are removed by, for example, filtration. A poor solvent such as methanol or hexane is added to the filtrate to precipitate the PHA. Furthermore, filtration or centrifugal separation is used to remove the supernatant. The remnant is then dried to collect the PHA.

The weight-average molecular weight (Mw) of the resultant PHA, and the composition of 3HH and other monomers (% by moles) therein can be analyzed by, for example, gel permeation chromatography, a gas chromatographic method or a nuclear magnetic resonance method.

### EXAMPLES

Hereinafter, the present invention will be described in detail by way of working examples thereof. However, the invention is not limited by these examples. General genetic manipulations can be performed as described in Molecular Cloning (Cold Spring Harbor Laboratory Press (1989)). Any enzyme, any cloning host, and any other that are used in the genetic manipulations are commercially available from suppliers in the market, and are usable in accordance with their manual. The enzyme is not particularly limited as long as the enzyme is an enzyme usable for genetic manipulation.

A KNK005ΔphaZ1,2,6 strain used in production examples, the working examples, and comparative examples described below is a transformant in which a phaZ1,2,6 gene on a chromosome of a C. necator H16 strain is deleted and to which a PHA synthase gene (gene encoding a PHA synthase having an amino acid sequence described in SEQ ID NO: 4) derived from Aeromonas caviae is introduced, and can be produced in accordance with a method in WO 2014/065253.

### (Production Example 1) Production of KNK005ΔphaZ1,2,6/nagEG793C,dR Strain

Initially, a plasmid for chromosome-substitution was produced. The production was performed as follows:

A chromosome DNA of a C. necator H16 strain was used as a template to perform a PCR, using primers described in SEQ ID NO: 8 and SEQ ID NO: 9. In the PCR, treatment at 98° C. was initially conducted for 2 minutes, and then the following were repeated up to 25 cycles: a series of reactions at 98° C. for 15 seconds, at 60° C. for 30 seconds, and at 68° C. for 2 minutes. A used polymerase was KOD-plus- (manufactured by Toyobo Co., Ltd.). In the same way, a PCR was performed, using primers described in SEQ ID NO: 10 and SEQ ID NO: 11. Furthermore, the two DNA fragments yielded in the PCRs were used as a template to perform a PCR under the same conditions using primers described in SEQ ID NOs: 8 and 11. The resultant DNA fragment was digested with a restriction enzyme SwaI. This DNA fragment was linked to a vector pNS2X-sacB digested with SwaI and described in JP 2007-259708 A through a DNA ligase (Ligation High (manufactured by Toyobo Co., Ltd.)) to produce a plasmid vector pNS2X-sacB+nagEG793C for chromosome-substitution having base sequences on the upstream side and the downstream side of the 793^{th} base of a nagF structural gene, and further containing a base sequence in which the base G at the 793^{th} base of the nagE structural gene was substituted with C.

Next, the plasmid vector pNS2X-sacB+nagEG793C for chromosome-substitution was used to produce a chromosome substituted strain KNK005ΔphaZ1,2,6/nagEG793C as described hereinafter.

The plasmid vector pNS2X-sacB+nagEG793C for chromosome-substitution was used to transform an E. coli S17-1 strain (ATCC47055). The resultant and a KNK005ΔphaZ1,2,6 strain were subjected to mixed culturing on a nutrient agar medium (manufactured by the company Difco) to attain a conjugative transfer.

The resultant cultured liquid was inoculated onto Simmons' agar medium containing 250 mg/L of kanamycin (2 g/L of sodium citrate, 5 g/L of sodium chloride, 0.2 g/L of magnesium sulfate heptahydrate, 1 g/L of ammonium dihydrogenphosphate, 1 g/L of potassium dihydrogenphosphate, and 15 g/L of agar; pH: 6.8). A bacterial strain which was growing on the agar medium was selected to gain a strain in which the above-mentioned plasmid was introduced to the chromosome of the KNK005ΔphaZ1,2,6 strain. This strain was subjected to two-generation culturing on a nutrient broth medium (manufactured by the company Difco), and then diluted and applied onto a nutrient agar medium containing 15% of sucrose. In this way, the bacterial strain which was growing was gained as a plasmid-dropped-out strain. Furthermore, one bacterial strain was isolated in which the 793^{th} base G of the nagE structural gene on the chromosome was substituted with C according to an analysis based on a DNA sequencer. This mutation introduced strain was named a KNK005ΔphaZ1,2,6/nagEG793C strain. The resultant KNK005ΔphaZ1,2,6/nagEG793C strain was a strain in or into which: a sequence from the initiation codon to the termination codon of each of phaZ76 and phaZ1 genes on the chromosome of the C. necator H16 strain was deleted; a sequence from the 16^{th} codon of a phaZ2 gene thereon to the termination codon thereof was deleted; a gene encoding a PHA synthase having an amino acid sequence described SEQ ID NO: 4 was introduced to the chromosome; and G which was the 793^{th} base of the nagE structural gene was substituted with C.

Furthermore, a plasmid for gene-disruption was produced. The production was performed as follows:

A chromosome DNA of a C. necator H16 strain was used as a template to perform a PCR, using primers described in SEQ ID NO: 12 and SEQ ID NO: 13. The PCR was performed under the same conditions as described above. A used polymerase was KOD-plus- (manufactured by Toyobo Co., Ltd.). In the same way, a PCR was performed, using primers described in SEQ ID NO: 14 and SEQ ID NO: 15. Furthermore, the two DNA fragments yielded in the PCRs were used as a template to perform a PCR under the same conditions, using primers described in SEQ ID NOs: 12 and 15. The resultant DNA fragment was digested with a restriction enzyme SwaI. This DNA was linked to a vector pNS2X-sacB digested with SwaI and described in JP 2007-259708 A through a DNA ligase (Ligation High (manufactured by Toyobo Co., Ltd.)) to produce a plasmid vector pNS2X-sacB+nagRUD for gene-disruption, having base sequences on the upstream side and the downstream side of a nagR structural gene.

Next, the plasmid vector pNS2X-sacB+nagRUD for gene-disruption was used to produce a gene disrupted strain KNK005ΔphaZ1,2,6/nagEG793C,dR strain as described hereinafter.

The plasmid vector pNS2X-sacB+nagRUD for gene-disruption was used to transform an E. coli S17-1 strain (ATCC47055). The resultant, and the KNK005ΔphaZ1,2,6/nagEG793C strain yielded as described above were subjected to mixed culturing on a nutrient agar medium (manufactured by the company Difco) to attain a conjugative transfer.

The resultant cultured liquid was inoculated onto Simmons' agar medium containing 250 mg/L of kanamycin (2 g/L of sodium citrate, 5 g/L of sodium chloride, 0.2 g/L of magnesium sulfate heptahydrate, 1 g/L of ammonium dihydrogenphosphate, 1 g/L of potassium dihydrogenphosphate, and 15 g/L of agar; pH: 6.8). A bacterial strain which was growing on the agar medium was selected to gain a strain in which the above-mentioned plasmid was introduced to the chromosome of the KNK005ΔphaZ1,2,6/nagEG793C strain. This strain was subjected to two-generation culturing on a nutrient broth medium (manufactured by the company Difco), and then diluted and applied onto a nutrient agar medium containing 15% of sucrose. In this way, the bacterial strain which was growing was gained as a plasmid-dropped-out strain. Furthermore, one bacterial strain was isolated in which a sequence from the initiation codon to the termination codon of the nagR gene on the chromosome was deleted according to an analysis based on a DNA sequencer. This gene disrupted strain was named a KNK005ΔphaZ1,2,6/nagEG793C,dR strain. The KNK005ΔphaZ1,2,6/nagEG793C,dR strain was a strain in or into which: the sequence from the initiation codon to the termination codon of each of the phaZ6 and phaZ1 genes on the chromosome of the C. necator H16 strain was deleted; the sequence from the 16^{th} codon of the phaZ2 gene thereon to the termination codon thereof was deleted; the gene encoding PHA synthase having the amino acid sequence described SEQ ID NO: 4 was introduced to the chromosome; G which was the 793^{th} base of the nagE structural gene was substituted with C; and further a sequence from the initiation codon to the terminal codon of the nagR gene was deleted.

### (Production Example 2) Production of pCUP2-lacUV5-cscAB in KNK005ΔphaZ1,2,6 Strain

Initially, a plasmid for expressing cscA and cscB genes was produced. The production was performed as follows:

An artificial gene synthesis was used to yield a plasmid into which a base sequence described in SEQ ID NO: 16 was introduced, which contained cscA and cscB genes. This plasmid was digested with restriction enzymes MunI and SpeI. The resultant DNA fragment containing the cscA and cscB genes was linked to a product in which a plasmid vector pCUP2 described in WO 2007/049716 was cut with MunI and Spa so as to yield a plasmid vector pCUP2-cscAB.

Furthermore, a genome DNA of an E. coli HB101 strain was used as a template to perform a PCR under the same conditions as in Production Example 1, using primers described in SEQ ID NO: 17 and SEQ ID NO: 18. The DNA fragment yielded by the PCR and containing a lacUV5 promoter was digested with MunI. This DNA fragment was linked to a product in which the plasmid vector pCUP2-cscAB was cut with MunI. From the resultant plasmids, a PCR was used to select a plasmid in which the DNA fiagment containing the lacUV5 promoter sequence was inserted in a direction along which cscA and cscB were positioned on the downstream side of a lacUV5 promoter. In this way, a plasmid vector pCUP2-lacUV5-cscAB was yielded.

Next, the plasmid vector pCUP2-lacUV5-cscAB was introduced to a KNK005ΔphaZ1,2,6 strain to yield a transformant pCUP2-lacUV5-cscAB in KNK005ΔphaZ1,2,6 strain.

The introduction of the plasmid vector into the cells was attained by electrical introduction as follows: A used gene introducing device was a gene pulser manufactured by Bio-Rad Laboratories, Inc., and a used cuvette was a cuvette having a gap of 0.2 cm and manufactured by the same incorporated company Bio-Rad Laboratories. Into the cuvette were injected 400 μL of the competent cells and 20 μL of the expression vector, and then the cuvette was set to the pulsing device to apply electric pulses thereto under conditions of an electrostatic capacitance of 25 μF, a voltage of 1.5 kV, and a resistance value of 800Ω. After the pulsing, the bacterial liquid in the cuvette was shaken and cultured on a nutrient broth medium (manufactured by a company Difco) at 30° C. for 3 hours. The bacterial liquid was cultured on a selection plate (nutrient agar medium (manufactured by the company Difco), using kanamycin (100 mg/L) at 30° C. for 2 days. A growing transformant pCUP2-lacUV5-cscAB in KNK005ΔphaZ1,2,6 was gained.

### (Production Example 3) Production of pCUP2-lacUV5-cscAB in KNK005ΔphaZ,1,2,6/nagEG793C,dR Strain

In the same way as in Production Example 2, the plasmid vector pCUP2-lacUV5-cscAB produced in Production Example 2 was introduced to the KNK005ΔphaZ1,2,6/nagEG793C,dR strain produced in Production Example 1, so as to yield a transformant pCUP2-lacUV5-cscAB in KNK005ΔphaZ1,2,6/nagEG793C,dR strain.

### (Production Example 4) Production of pCUP2-lacUV5-cscA in KNK005ΔphaZ1,2,6 Strain

Initially, a plasmid for expressing a cscA gene was produced. The production was performed as follows:

The plasmid vector pCUP2-lacUV5-cscAB described in Production Example 2 was used as a template to perform a PCR under the same conditions as in Production Example 1, using primers described in SEQ ID NO: 19 and SEQ ID NO: 20. The DNA fragment yielded by the PCR and containing a cscA gene sequence was digested with restriction enzymes MunI and SpeI. The resultant DNA was linked to a product in which a plasmid vector pCUP2 described in WO 2007/049716 was cut with MunI and SpeI, so as to yield a plasmid vector pCUP2-cscA. Furthermore, a genome DNA of an E. coli HB101 strain was used as a template to perform a PCR under the same conditions as in Production Example 1, using primers described in SEQ ID NO: 17 and SEQ ID NO: 18. The DNA fragment yielded by the PCR and containing a lacUV5 promoter was digested with MunI. This DNA fragment was linked to a product in which the above-mentioned plasmid vector pCUP2-cscA was cut with MunI. From the resultant plasmids, a PCR was used to select a plasmid in which the DNA fragment containing the lacUV5 promoter sequence was inserted in a direction along which cscA was positioned on the downstream side of the lacUV5 promoter. In this way, a plasmid vector pCUP2-lacUV5-cscA was yielded.

Next, in the same way as in Production Example 2, the plasmid vector pCUP2-lacUV5-cscA was introduced to a KNK005ΔphaZ1,2,6 strain, so as to yield a transformant pCUP2-lacUV5-cscA in KNK005ΔphaZ1,2,6 strain.

### (Production Example 5) Production of pCUP2-lacUV5-cscB in KNK005ΔphaZ1,2,6 Strain

Initially, a plasmid for expressing a cscB gene was produced. The production was performed as follows:

The plasmid vector pCUP2-lacUV5-cscAB described in Production Example 2 was used as a template to perform a PCR under the same conditions as in Production Example 1, using primers described in SEQ ID NO: 21 and SEQ ID NO: 22. A DNA fragment yielded by the PCR and containing a cscB gene sequence was digested with restriction enzymes MunI and SpeI. The resultant was linked to a product in which a plasmid vector pCUP2 described in WO 2007/049716 was cut with MunI and SpeI, so as to yield a plasmid vector pCUP2-cscB. Furthermore, a genome DNA of an E. coli HB101 strain was used as a template to perform a PCR under the same conditions as in Production Example 1, using primers described in SEQ ID NO: 17 and SEQ ID NO: 18. The DNA fragment yielded by the PCR and containing a lacUV5 promoter was digested with MunI. This DNA fragment was linked to a product in which the above-mentioned plasmid vector pCUP2-cscB was cut with MunI. From the resultant plasmids, a PCR was used to select a plasmid in which the DNA fragment containing the lacUV5 promoter sequence was inserted in a direction along which cscB was positioned on the downstream side of the lacUV5 promoter. In this way, a plasmid vector pCUP2-lacUV5-cscB was yielded.

Next, in the same way as in Production Example 2, the plasmid vector pCUP2-lacUV5-cscB was introduced to a KNK005ΔphaZ1,2,6 strain, so as to yield a transformant pCUP2-lacUV5-cscB in KNK005ΔphaZ1,2,6 strain.

### (Production Example 6) Production of pCUP2-lacUV5-cscAB in KNK144S Strain

Initially, a plasmid bAO/pBlu/SacB-Km described in JP 2013-9627 A was used to produce a promoter and ribosome-binding-sequence inserted strain ACP-bktB/ΔphaZ1,2,6/nagEG793C,dR strain as follows:

A plasmid bAO/pBlu/SacB-Km was used to transform an E. coli S17-1 strain (ATCC47055). The resultant and the KNK005ΔphaZ1,2,6/nagEG793C,dR strain yielded in Production Example 1 were subjected to mixed culturing on a nutrient agar medium (manufactured by the company Difco) to attain a conjugative transfer.

The resultant cultured liquid was inoculated onto Simmons' agar medium containing 250 mg/L of kanamycin (2 g/L of sodium citrate, 5 g/L of sodium chloride, 0.2 g/L of magnesium sulfate heptahydrate, 1 g/L of ammonium dihydrogenphosphate, 1 g/L of potassium dihydrogenphosphate, and 15 g/L of agar; pH: 6.8). A bacterial strain which was growing on the agar medium was selected to gain a strain in which the above-mentioned plasmid was introduced to the chromosome of the KNK005ΔphaZ1,2,6/nagEG793C,dR strain. This strain was subjected to two-generation culturing on a nutrient broth medium (manufactured by the company Difco), and then diluted and applied onto a nutrient agar medium containing 15% of sucrose. In this way, the bacterial strain which was growing was gained as a plasmid-dropped-out strain. Furthermore, one bacterial strain was isolated in which DNA made of a base sequence containing a promoter of a phaC gene of A. caviae and a ribosome binding sequence was inserted to a position immediately before the initiation codon of a bktB gene of the chromosome according to an analysis based on a DNA sequencer. This gene inserted strain was named an ACP-bktB/ΔphaZ1,2,6/nagEG793C,dR strain. The ACP-bktB/ΔphaZ1,2,6/nagEG793C,dR strain was a strain in or into which: a sequence from the initiation codon to the termination codon of each of phaZ6 and phaZ1 genes on the chromosome of the C. necator H16 strain was deleted; a sequence from the 16^{th} codon of a phaZ2 gene thereon to the termination codon thereof was deleted; a gene encoding a PHA synthase having an amino acid sequence described SEQ ID NO: 4 was introduced to the chromosome; G which was the 793^{th} base of a nagE structural gene was substituted with C; a sequence from the initiation codon to the termination codon of a nagR gene was deleted; and further DNA made of a base sequence containing the promoter of the phaC gene of A. caviae and the ribosome binding sequence was inserted to the position immediately before the initiation codon of a bktB (13 ketothiolase) gene.

Furthermore, a promoter and ribosome-binding-sequence inserted strain ACP-bktB/ΔphaZ1,2,6/nagEG793C,dR/trc-J4b strain was produced as follows:

Initially, a plasmid for promoter and ribosome-binding-sequence insertion was produced. The production was performed as follows:

A chromosome DNA of a C. necator H16 strain was used as a template to perform a PCR under the same conditions as in Production Example 1, using primers described in SEQ ID NO: 23 and SEQ ID NO: 24. In the same way, a PCR was performed under the same conditions, using primers described in SEQ ID NO: 25 and SEQ ID NO: 26. Furthermore, a plasmid pKK388-1 (manufactured by Clontech Laboratories, Inc.) was used as a template to perform a PCR under the same conditions, using primers described in SEQ ID NO: 27 and SEQ ID NO: 28. The three DNA fragments yielded in the PCRs were used as a template to perform a PCR under the same conditions, using primers described in SEQ ID NOs: 23 and 26. The resultant DNA fragment was digested with a restriction enzyme SwaI. This DNA fragment was linked to a vector pNS2X-sacB digested with SwaI and described in JP 2007-259708 A through a DNA ligase (Ligation High (manufactured by Toyobo Co., Ltd.)) to produce a plasmid vector pNS2X-sacB+phaJ4bU-trc-phaJ4b for promoter and ribosome-binding-sequence insertion, having a base sequence on the upstream side of a phaJ4b structural gene, a trc promoter, a ribosome binding sequence, and a phaJ4b structural gene sequence.

Next, the plasmid vector pNS2X-sacB+phaJ4bU-trc-phaJ4b for promoter and ribosome-binding-sequence insertion, was used to perform a conjugative transfer, a selection on Simmons'agar medium, and a selection on a nutrient agar medium containing 15% of sucrose in the same way as used to produce the above-mentioned promoter and ribosome-binding-sequence inserted strain, using the ACP-bktB/ΔphaZ1,2,6/nagEG793C,dR strain as a parent strain. In this way, an ACP-bktB/ΔphaZ1,2,6/nagEG793C,dR/trc-J4b strain was produced. The ACP-bktB/ΔphaZ1,2,6/nagEG793C,dR/trc-J4b strain was a strain in or into which: the sequence from the initiation codon to the termination codon of each of the phaZ6 and phaZ1 genes on the chromosome of the C. necator H16 strain was deleted; the sequence from the 16^{th} codon of the phaZ2 gene to the termination codon thereof was deleted; the gene encoding the PHA synthase having the amino acid sequence described SEQ ID NO: 4 was introduced to the chromosome; G which was the 793^{th} base of the nagE structural gene was substituted with C; the sequence from the initiation codon to the terminal codon of the nagR gene was deleted; DNA made of the base sequence containing the promoter of the phaC gene of A. caviae, and the ribosome binding sequence was inserted to the position immediately before the initiation codon of the bktB gene; and further DNA made of a base sequence containing the trc promoter and the ribosome binding sequence was inserted to the position immediately before the initiation codon of the phaJ4b gene.

Furthermore, a chromosome substituted strain KNK144S strain was produced as follows:

A vector pBlueASRU for chromosome-substitution, described in JP2008-29218 A was used to perform a conjugative transfer, a selection on Simmons' agar medium, and a selection on a nutrient agar medium containing 15% of sucrose in the same way as used to produce the chromosome substituted strain described in Production Example 1, using the ACP-bktB/ΔphaZ1,2,6/nagEG793C,dR/trc-J4b strain as a parent strain. In this way, a KNK144S strain was produced. The KNK144S strain was a strain in or into which: the sequence from the initiation codon to the termination codon of each of the phaZ6 and phaZ1 genes on the chromosome of the C. necator H16 strain was deleted; the sequence from the 16^{th} codon of the phaZ2 gene thereon to the termination codon thereof was deleted; the gene encoding the PHA synthase having the amino acid sequence described SEQ ID NO: 4 was introduced to the chromosome; G which was the 793^{th} base of the na F structural gene was substituted with C; the sequence from the initiation codon to the terminal codon of the nagR gene was deleted; DNA made of the base sequence containing the promoter of the phaC gene of A. caviae, and the ribosome binding sequence was inserted to the position immediately before the initiation codon of the bktB gene; DNA made of the base sequence containing the trc promoter and the ribosome binding sequence was inserted to the position immediately before the initiation codon of the phaJ4b gene; and further a terminal codon and an NheI cut moiety were produced in a phaA structural gene sequence.

Next, in the same way as in Production Example 2, the plasmid vector pCUP2-lacUV5-cscAB described in Production Example 2 was introduced to the KNK144S strain, so as to yield a transformant pCUP2-lacUV5-cscAB in KNK144S strain.

### (Production Example 7) Production of pCUP2-lacUV5-cscAB in KNK143S Strain

Initially, a plasmid was produced for promoter, ribosome-binding-sequence, and gene insertions. The production was performed as follows:

A chromosome DNA of a C. necator H16 strain was used as a template to perform a PCR, using primers described in SEQ ID NO: 29 and SEQ ID NO: 30. The PCR was performed under the same conditions as described above. A used polymerase was KOD-plus- (manufactured by Toyobo Co., Ltd.). In the same way, a PCR was performed, using primers described in SEQ ID NO: 31 and SEQ ID NO: 32. Furthermore, the two DNA fragments yielded in the PCRs were used as a template to perform a PCR under the same conditions, using primers described in SEQ ID NOs: 29 and 32. The resultant DNA fragment was digested with a restriction enzyme SwaI. This DNA fragment was linked to a vector pNS2X-sacB digested with SwaI and described in JP 2007-259708 A through a DNA ligase (Ligation High (manufactured by Toyobo Co., Ltd.)) to produce a plasmid vector pNS2X-sacB+phaZ2MunISpeI having base sequences on the upstream side and the downstream side of a phaZ2 structural gene.

Next, an artificial gene synthesis was used, so as to yield a plasmid into which a base sequence containing a ribosome binding sequence, ccr and emd, this base sequence being described in SEQ ID NO: 33, was introduced. This plasmid was digested with restriction enzymes MunI and SpeI. The resultant DNA fragment containing the ribosome binding sequence, and the ccr and emd genes was linked to a product in which the plasmid vector pNS2X-sacB+Z2UDMunISpeI was cut with MunI and SpeI, so as to yield a plasmid vector pNS2X-sacB+Z2U-ccr-emd-Z2D.

Next, a plasmid pKK388-1 (manufactured by Clontcch Laboratories, Inc.) was used as a template to perform a PCR under the same conditions, using primers described in SEQ ID NO: 34 and SEQ ID NO: 35. The DNA fragment yielded by the PCR and containing a trc promoter was digested with MunI. This DNA fragment was linked to a product in which the above-mentioned plasmid vector pNS2X-sacB+Z2U-ccr-emd-Z2D was cut with MunI. From the resultant plasmids, a PCR was used to select a plasmid in which the DNA fragment containing the trc promoter sequence was inserted in a direction along which ccr and emd were positioned on the downstream side of the trc promoter. In this way, a plasmid vector pNS2X-sacB+Z2U-trc-ccr-emd-Z2D was yielded for promoter, ribosome-binding-sequence, and gene insertions.

Next, the plasmid vector pNS2X-sacB+Z2U-trc-ccr-emd-Z2D for promoter, ribosome-binding-sequence, and gene insertions was used to perform a conjugative transfer, a selection on Simmons' agar medium, and a selection on a nutrient agar medium containing 15% of sucrose in the same way as used to produce the promoter and ribosome-binding-sequence inserted strain described in Production Example 6, using a KNKPVIS strain as a parent strain. In this way, a KNK143S strain was produced. The KNK143S strain was a strain in or into which: a sequence from the initiation codon to the termination codon of each of phaZ6 and phaZ1 genes on the chromosome of the C. necator 1-116 strain was deleted; a sequence from the 16^{th} codon of a phaZ2 gene thereon to the termination codon thereof was deleted; a gene encoding a PHA synthase having an amino acid sequence described SEQ ID NO: 4 was introduced to the chromosome; G which was the 793^{th} base of a nagE structural gene was substituted with C; a sequence from the initiation codon to the terminal codon of a nagR gene was deleted; DNA made of a base sequence containing a promoter of a phaC gene of A. caviae, and a ribosome binding sequence was inserted to a position immediately before the initiation codon of a bktB gene; DNA made of a base sequence containing the trc promoter and a ribosome binding sequence was inserted to the position immediately before the initiation codon of a phaJ4b gene; a termination codon and a restriction enzyme NheI cut moiety were produced in a phaA structural gene sequence; and the trc promoter, a ribosome binding sequence, and the ccr and emd genes were inserted to the position where the phaZ2 gene was originally present.

Next, in the same way as in Production Example 2, the plasmid vector pCUP2-lacUV5-cscAB described in Production Example 2 was introduced to the KNK143S strain, so as to yield a transformant pCUP2-lacUV5-cscAB in KNK143S strain.

### (Production Example 8) Production of pCUP2-lacUV5-cscAB in KNK140S Strain

Initially, a plasmid for gene-disruption was produced. The production was performed as follows:

A chromosome DNA of a KNK005ΔphaZ1,2,6 strain was used as a template to perform a PCR under the same conditions as in Production Example 1, using primers described in SEQ ID NO: 36 and SEQ ID NO: 37. In the same way, a PCR was performed under the same conditions, using primers described in SEQ ID) NO: 38 and SEQ ID NO: 39. The two DNA fragments yielded in the PCRs were used as a template to perform a PCR under the same conditions, using primers described in SEQ ID NOs: 36 and 39. The resultant DNA fragment was digested with a restriction enzyme SwaI. This DNA fragment was linked to a vector pNS2X-sacB digested with SwaI and described in JP 2007-259708 A through a DNA ligase (Ligation High (manufactured by Toyobo Co., Ltd.)) to produce a plasmid vector pNS2X-sacB+phaAB1UD for gene-disruption, having a base sequence on the upstream side of a phaA structural gene, and a base sequence on the downstream side of a phaB1 (acetoacetyl CoA reductase) structural gene.

Next, the plasmid vector pNS2X-sacB+phaAB1UD for gene-disruption was used to perform a conjugative transfer, a selection on Simmons' agar medium, and a selection on a nutrient agar medium containing 15% of sucrose in the same way as used to produce the strain for chromosome-substitution, described in Production Example 1, using a KNK144S strain as a parent strain. In this way, a KNK140S strain was produced. The KNK140S strain was a strain in or into which: a sequence from the initiation codon to the termination codon of each of phaZ6 and phaZ1 genes on the chromosome of the C. necator H16 strain was deleted; a sequence from the 16^{th} codon of a phaZ2 gene thereon to the termination codon thereof was deleted; a gene encoding a PHA synthase having an amino acid sequence described SEQ ID NO: 4 was introduced to the chromosome; G which was the 793^{th} base of a nagE structural gene was substituted with C; a sequence from the initiation codon to the terminal codon of a nagR gene was deleted; DNA made of the base sequence containing the promoter of the phaC gene of A. caviae, and a ribosome binding sequence was inserted to the position immediately before the initiation codon of a bktB gene; DNA made of a base sequence containing a trc promoter and a ribosome binding sequence was inserted to the position immediately before the initiation codon of a phaJ4b gene; and further a sequence from the initiation codon of the phaA gene to a termination codon of the phaB1 was deleted.

Next, in the same way as in Production Example 2, the plasmid vector pCUP2-lacUV5-cscAB described in Production Example 2 was introduced to the KNK140S strain, so as to yield a transformant pCUP2-lacUV5-cscAB in KNK140S strain.

### (Production Example 9) Production of pCUP2-lacUV5-cscAB in KNK142S Strain

The plasmid vector pNS2X-sacB+phaAB1UD for gene-disruption, described in Production Example 8 was used to perform a conjugative transfer, a selection on Simmons' agar medium, and a selection on a nutrient agar medium containing 15% of sucrose in the same way as used to produce the strain for chromosome-substitution, described in Production Example 1, using the KNK-143S strain described in Production Example 7 as a parent strain. In this way, a KNK142S strain was produced. The KNK142S strain was a strain in or into which: a sequence from the initiation codon to the termination codon of each of phaZ6 and phaZ1 genes on the chromosome of the C. necator H16 strain was deleted; a sequence from the 16^{th} codon of a phaZ2 gene thereon to the termination codon thereof was deleted; a gene encoding a PHA synthase having an amino acid sequence described SEQ ID NO: 4 was introduced to the chromosome; G which was the 793^{th} base of a nagE structural gene was substituted with C; a sequence from the initiation codon to the terminal codon of a nagR gene was deleted; DNA made of a base sequence containing a promoter of a phaC gene of A. caviae, and a ribosome binding sequence was inserted to the position immediately before the initiation codon of a bktB gene; DNA made of a base sequence containing a trc promoter and a ribosome binding sequence was inserted to the position immediately before the initiation codon of a phaJ4b; a trc promoter, a ribosome binding sequence, and ccr and emd genes were inserted to the position where the phaZ2 gene was originally present; and further a sequence from the initiation codon of a phaA gene to a termination codon of a phaB1 was deleted.

Next, in the same way as in Production Example 2, the plasmid vector pCUP2-lacUV5-cscAB described in Production Example 2 was introduced to the KNK142S strain, so as to yield a transformant pCUP2-lacUV5-cscAB in KNK142S strain.

### (Comparative Examples 1 to 4) Sucrose Assimilability of Each of KNK005ΔphaZ1,2,6 Strain, KNK005ΔphaZ1,2,6/nagEG793C,dR Strain, pCUP2-lacUV5-cscA in KNK005ΔphaZ1,2,6 Strain, and pCUP2-lacUV5-cscB in KNK005ΔphaZ1,2,6 Strain; PHA Producing Performance Thereof; and 3HH Composition Proportion in PHA

In each of the examples, the composition of a seed medium was set as follows: 1 w/v % of a meat-extract, 1 w/v % of bacto-trypton, 0.2 w/v % of a yeast-extract, 0.9 w/v % of Na₂HPO₄.12H₂O, and 0.15 w/v % KH₂PO₄. When the plasmid vector introduced strain of the example was cultured in the seed medium, kanamycin was added to the seed medium to give a final concentration of 100 μg/mL.

A producing medium used for a sucrose assimilability test and PHA production was set as follows: 1.1 w/v % of Na₂HPO₄.12H₂O, 0.19 w/v % of KH₂PO₄, 0.13 w/v % of (NH₄)₂SO₄, 0.1 w/v % of MgSO₄.7H₂O, 0.1 v/v % of trace metal salt solution (solution in which into a 0.1 N solution of hydrochloric acid were dissolved 1.6 w/v % of FeCl₃.6H₂O, 1 w/v % of CaCl₂.2H₂O, 0.02 w/v % of CoCl₂.6H₂O, 0.016 w/v % of CuSO₄.5H₂O, and 0.012 w/v % of NiCl₂.6H₂O. A used carbon source was a single carbon source of a 40 w/v % of aqueous sucrose solution, and this solution was added to the medium to give a concentration of 1.5 w/v %.

The following was inoculated into the seed medium (5 mL): a glycerol stock (50 μL) of each of a KNK005ΔphaZ1,2,6 strain (see WO 2014/065253), the KNK005ΔphaZ1,2,6/nagEG793C,dR strain produced in Production Example 1, the pCUP2-lacUV5-cscA in KNK005ΔphaZ1,2,6 strain produced in Production Example 4, and the pCUP2-lacUV5-cscB in KNK005ΔphaZ1,2,6 strain produced in Production Example 5. The resultant was shaken and cultured at 30° C. for 24 hours. The resultant cultured liquid was used as a seed.

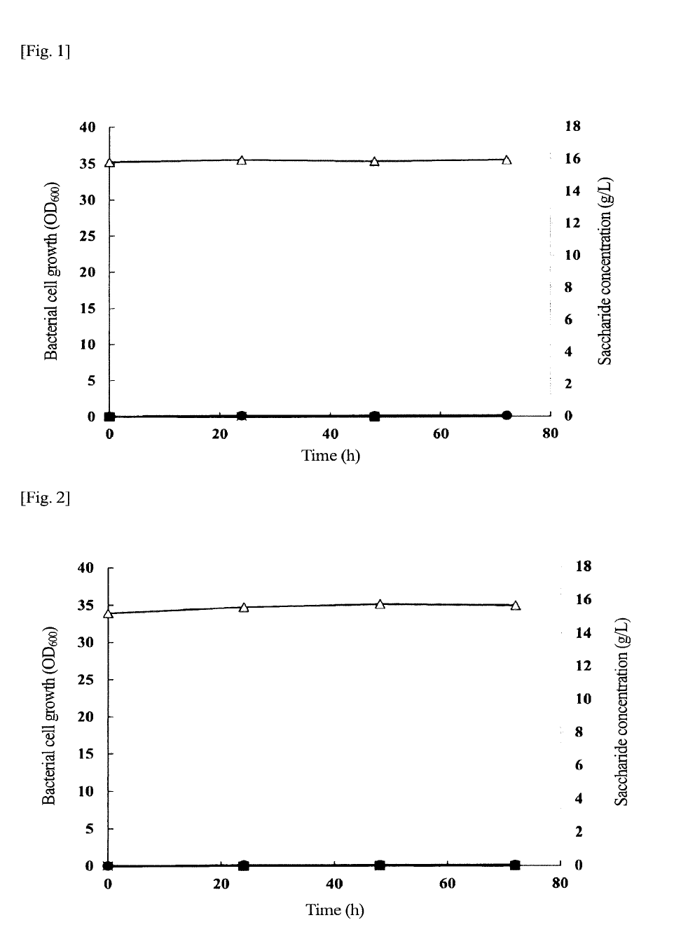

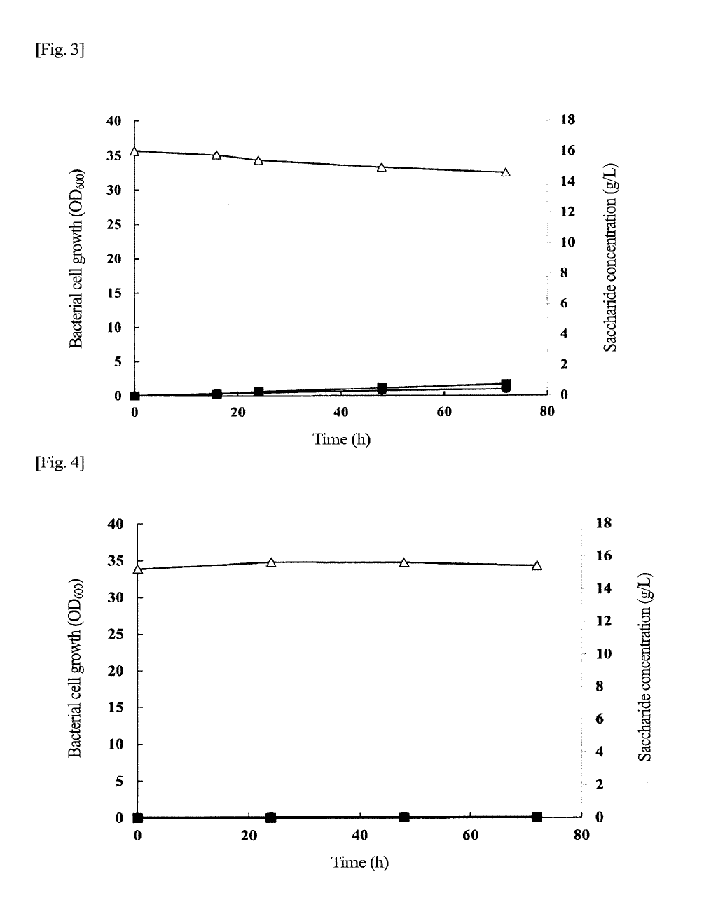

In the sucrose assimilability test and PHA producing culturing, the seed was inoculated in an amount of 1.0 v/v %, into Sakaguchi flask in which 200 mL of the producing medium was put, and the seed was shaken and cultured at a culturing temperature of 30° C. With time, the cultured liquid was sampled, and measurements were made about the growth (OD₆₀₀) of bacterial cells therein, and the concentration of each of saccharides (sucrose, glucose and fructose) in the medium. The measurement of the concentration of the saccharide was made, using an “F-Kit Sucrose/D-Glucose/Fructose” (manufactured by J.K. International, Inc.). The results are shown in FIGS. 1 to 4.

The bacterial cells were cultured for 72 hours. Thereafter, the cells were collected by centrifugal separation, washed with methanol, and freeze-dried. The weight of the dried bacterial cells was then measured.

The PHA production amount was calculated out as follows: To the resultant dry bacterial cells was added chloroform in an amount of 100 mL, per one gram of the cells. At room temperature, the resultant was stirred a whole day and night. Any PHA in the bacterial cells was extracted. The bacterial cell residue was filtrated away, and an evaporator was used to concentrate the residue until the total volume thereof turned into ⅓. Thereto was gradually added hexane in a volume three times the concentrated liquid volume. The liquid was allowed to stand still for 1 hour while slowly stirred. The precipitated PHA was filtrated away, and the PHA was vacuum-dried at 50° C. for 3 hours. The weight of the dried PHA was measured to calculate out the PHA production amount. The results are shown in Table 1.

The 3HH composition proportion in the produced PHA was measured by gas chromatography as follows: To about 20 mg of the dried PHA were added 2 mL of a sulfuric-acid/methanol mixed solution (15/85) and 2 mL of chloroform, and the system was airtightly sealed. The system was heated at 100° C. for 140 minutes to yield a methyl ester of a PHA decomposed product. After the system was cooled, to this product was added 1.5 g of sodium hydrogencarbonate bit by bit to neutralize the product. The system was allowed to stand still until the generation of carbon dioxide gas was stopped. Thereto was added 4 mL of diisopropyl ether, and the entire components were sufficiently mixed with each other. Thereafter, the resultant was subjected to centrifugal separation. A capillary gas chromatography was used to analyze the monomer unit composition of the PHA decomposed product in the supernatant. The used gas chromatograph was an instrument GC-17A manufactured by Shimadzu Corporation, and the used capillary column was a column NEUTRA BOND-1 (column length: 25 m, column inside diameter: 0.25 mm, and a liquid membrane thickness: 0.4 μm) manufactured by GL Sciences Inc. The used carrier gas was He, and the column inlet pressure was set to 100 kPa. Any sample was injected in a volume of 1 μL. The temperature conditions were as follows: the temperature was raised at a rate of 8° C./minute from a starting temperature of 100° C. to 200° C., and the temperature was raised at a rate of 30° C./minute from 200° C. to 290° C. Under the above-mentioned conditions, the analysis was made. The resultant composition proportion of 3HH in the PHA is shown in Table 1.

The respective KNK005ΔphaZ1,2,6 strain, KNK005ΔphaZ1,2,6/nagEG793C,dR strain and pCUP2-lacUV5-cscB in KNK005ΔphaZ1,2,6 strain in Comparative Examples 1, 2 and 4 failed to proliferate, using sucrose as a carbon source. The pCUP2-lacUV5-cscA in KNK005ΔphaZ1,2,6 strain of Comparative Example 3 made use of sucrose as a carbon source to proliferate slightly. However, the growth speed thereof was very small. The PHA produced by the pCUP2-lacUV5-cscA in KNK005ΔphaZ1,2,6 strain of Comparative Example 3 contained no 3HH monomer units. The PHA was PHB, which is a homopolymer of 3HB.

### (Example 1) Sucrose Assimilability of pCUP2-lacUV5-cscAB in KNK005ΔphaZ,1,2,6 Strain; PHA Producing Performance thereof; and 3HH Composition Proportion in PHA

The composition of a seed medium was rendered the same as described in each of Comparative Examples 1 to 4. When the plasmid vector introduced strain of the example was cultured in the seed medium, kanamycin was added to the seed medium to give a final concentration of 100 μg/mL.

The composition of a producing medium used in a sucrose assimilability test and PHA production, and a carbon source used therein were rendered the same as described in each of Comparative Examples 1 to 4.

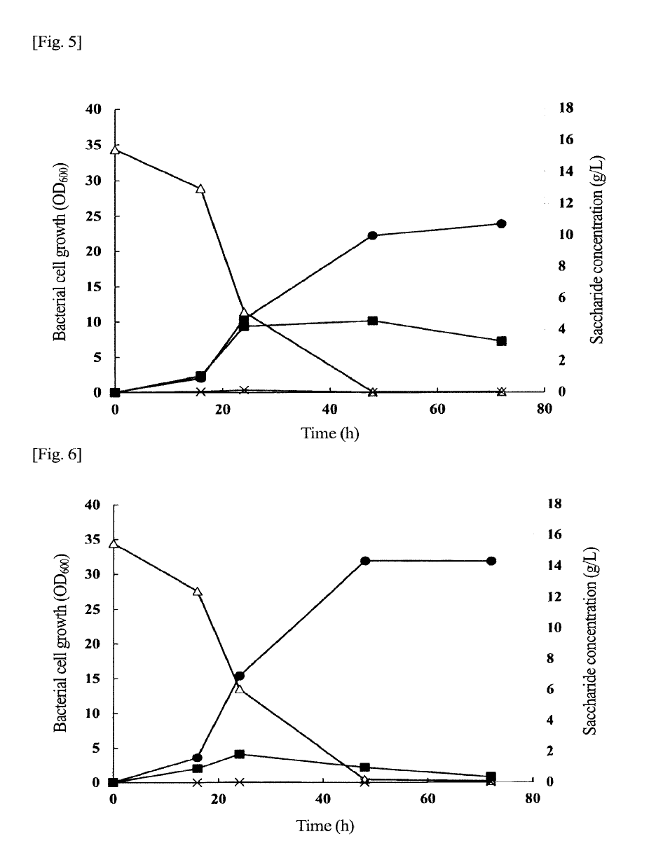

The pCUP2-lacUV5-cscAB in KNK005ΔphaZ1,2,6 strain produced in Production Example 2 was cultured in the same way as in Comparative Examples 1 to 4. With time, the cultured liquid was sampled, and measurements were made about the growth (OD₆₀₀) of bacterial cells therein, and the concentration of each of saccharides (sucrose, glucose, and fructose) in the medium. The measurement of the concentration of each of the saccharides was made in the same way as in Comparative Examples 1 to 4. The results are shown in FIG. 5.

The bacterial cells were cultured for 72 hours. Thereafter, the cells were collected by centrifugal separation, washed with methanol, and freeze-dried. The weight of the dried bacterial cells was then measured.

The PHA production amount and the 3HH composition proportion were calculated out in the same way as in Comparative Examples 1 to 4. The resultant PHA production amount and composition proportion of 3HH are shown in Table 1.

The pCUP2-lacUV5-cscAB in KNK005ΔphaZ1,2,6 strain made use of sucrose as a carbon source to proliferate satisfactorily to produce a PHA. The produced PHA was PHB, which is a homopolymer.

### (Example 2) Sucrose, Glucose, and Fructose Assimilabilities of pCUP2-lacUV5-cscAB in KNK005ΔphaZ1,2,6/nagEG793C,dR Strain; PHA Producing Performance Thereof; and 3HH Composition Proportion in PHA

The composition of a seed medium was rendered the same as described in each of Comparative Examples 1 to 4. When the plasmid vector introduced strain of the example was cultured in the seed medium, kanamycin was added to the seed medium to give a final concentration of 100 μg/mL.

The composition of a producing medium used in a sucrose assimilability test and PHA production were rendered the same as described in each of Comparative Examples 1 to 4. A used carbon source was a single carbon source of a 40 w/v % of aqueous sucrose solution, and this solution was added to the medium to give a concentration of 1.5 w/v %.

The pCUP2-lacUV5-cscAB in KNK005ΔphaZ1,2,6/nagEG793C,dR strain produced in Production Example 3 was cultured in the same way as in Comparative Examples 1 to 4. With time, the cultured liquid was sampled, and measurements were made about the growth (OD₆₀₀) of bacterial cells therein, and the concentration of each of saccharides (sucrose, glucose and fructose) in the medium. The measurement of the concentration of each of the saccharides was made in the same way as in Comparative Examples 1 to 4. The results are shown in FIG. 6.

The bacterial cells were cultured for 72 hours. Thereafter, the cells were collected by centrifugal separation, washed with methanol, and freeze-dried. The weight of the dried bacterial cells was then measured.

The PHA production amount and the 3HH composition proportion were calculated out in the same way as in Comparative Examples 1 to 4. The results are shown in Table 1.

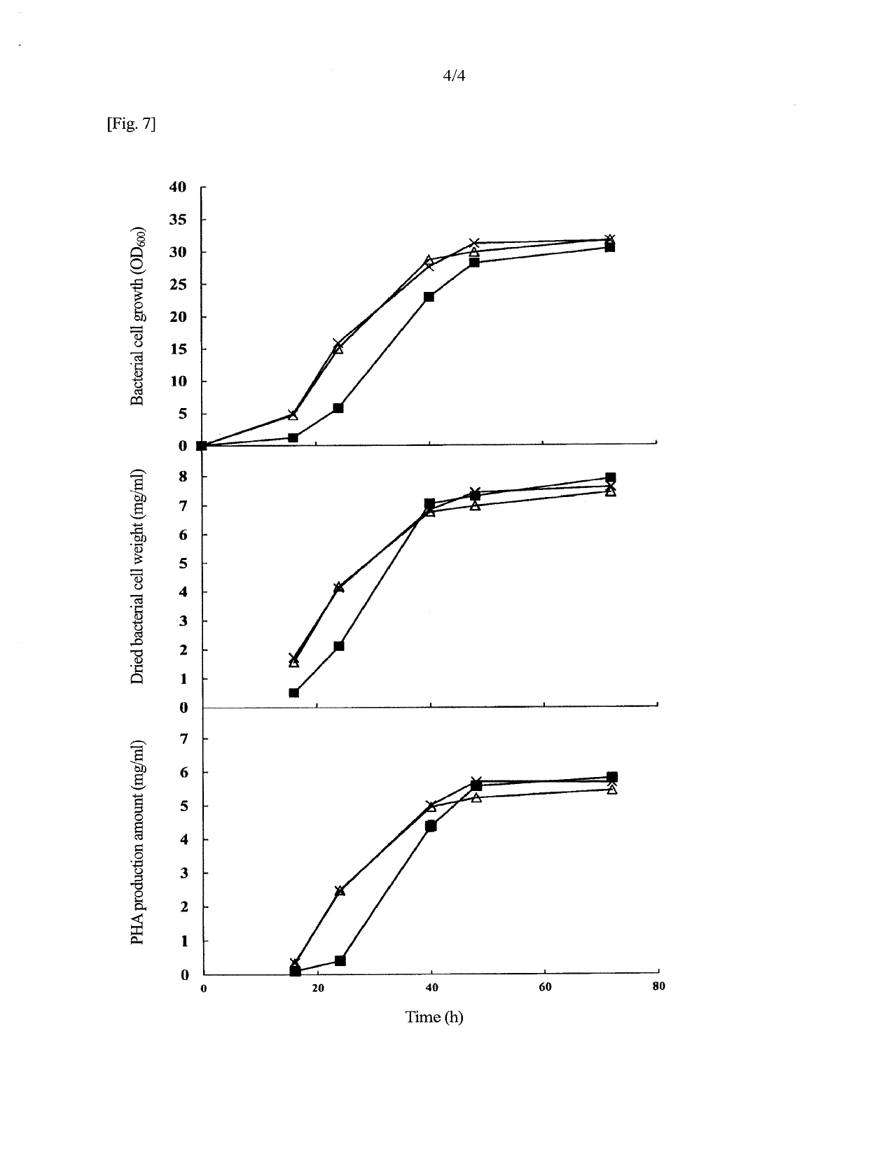

Under the same conditions as described above, culturing was performed in a producing medium in which the carbon source was changed from the aqueous sucrose solution to an aqueous solution of glucose or fructose that had the same concentration. With time, the cultured liquid was sampled, and measurements were made about the growth (OD₆₀₀) of bacterial cells therein, the weight of the dried bacterial cells, and the amount of a produced PHA. The results are shown in FIG. 7.

The 3HH composition proportion in the produced PHA was calculated out in the same way as in Comparative Examples 1 to 4. The composition proportion of 3HH in the resultant PHA is shown in Table 1.

The pCUP2-lacUV5-cscAB in KNK005ΔphaZ1,2,6/nagEG793C,dR strain made use of sucrose as a carbon source to show an especially good growth potential and PHA producing performance. When sucrose was used as the carbon source, the growth rate was larger than when glucose was used as the carbon source, and was equivalent to that when fructose, which C. necator 1116 strains can originally assimilate, was used as the carbon source.

The PHA produced by the pCUP2-lacUV5-cscAB in KNK005ΔphaZ1,2,6/nagEG793C,dR strain was PHB, which is a homopolymer of 3HB.

### (Examples 3 to 6) PHA Producing Performance of Each of pCUP2-lacUV5-cscAB KNK144S Strain, pCUP2-lacUV5-cscAB in KNK143S Strain, pCUP2-lacUV5-cscAB in KNK140S Strain, and pCUP2-lacUV5-cscAB iii KNK142S Strain; and 3HH Composition Proportion in PHA

The composition of a seed medium was rendered the same as described in each of Comparative Examples 1 to 4. When the plasmid vector introduced strain of the example was cultured in the seed medium, kanamycin was added to the seed medium to give a final concentration of 100 μg/mL.

The composition of a producing medium used for PHA production, and a carbon source used therein were rendered the same as described in each of Comparative Examples 1 to 4. The pCUP2-lacUV5-cscAB in KNK144S strain, the pCUP2-lacUV5-cscAB in KNK143S strain, the pCUP2-lacUV5-cscAB in KNK140S strain, and the pCUP2-lacUV5-cscAB in KNK142S strain produced, respectively, in Production Examples 6 to 9 were each cultured in the same way as in Comparative Examples 1 to 4. After the culturing for 72 hours, the resultant bacterial cells were collected by centrifugal separation, washed with methanol, and freeze-dried. The weight of the dried bacterial cells was measured.

The PHA production amount and the 3HH composition proportion were calculated out in the same way as in Comparative Examples 1 to 4. The results are shown in Table 1.

Each of the strains made use of sucrose as a carbon source to proliferate satisfactorily to produce a PHA. The PHA produced by each of the pCUP2-lacUV5-cscAB in KNK144S strain in Example 3, and the pCUP2-lacUV5-cscAB in KNK140S strain in Example 5 was PHBH containing a slight amount of the monomer of 3HH. The PHA produced by the pCUP2-lacUV5-cscAB in KNK143S strain in Example 4 was PHBH in which the composition proportion of 3HH was 2.3%. The PHA produced by the pCUP2-lacUV5-cscAB in KNK142S strain of Example 6 was PHBH, which the composition proportion of 3HH was a very high value of 26.7%.

TABLE 1

| | | | PHA production | 3HH composition |
|---|---|---|---|---|
| | | Introduced or enhanced | amount | proportion |
| | Bacterial strain name | gene(s) or property | (g/L) | (% by mole) |
| Example 1 | pCUP2-lacUV5-cscAB in KNK005 [symbol] phaZ 1, 2, 6 | cscA + cscB | 3.2 | 0 |
| | (Production Example 2) | | | |
| Example 2 | pCUP2-lacUV5-cscAB in KNK005 [symbol] phaZ 1, 2, 6/ | Glucose assimilability + | 5.1 | 0 |
| | nagEG793C, dR (Production Example 3) | cscA + cscB | | |
| Example 3 | pCUP2-lacUV5-cscAB in KNK144S | Glucose assimilability + | 4.8 | 0.1 |
| | (Production Example 6) | cscA + cscB + phaJ4b | | |
| Example 4 | pCUP2-lacUV5-cscAB in KNK143S | Glucose assimilability + | 4.5 | 2.3 |
| | (Production Example 7) | cscA + cscB + phaJ4b + | | |
| | | ccr + emd | | |
| Example 5 | pCUP2-lacUV5-cscAB in KNK140S | Glucose assimilability + | 3.1 | 0.2 |
| | (Production Example 8) | cscA + cscB + phaJ4b- | | |
| | | phaB1 | | |
| Example 6 | pCUP2-lacUV5-cscAB in KNK142S | Glucose assimilability + | 2.7 | 26.7 |
| | (Production Example 9) | cscA + cscB + phaJ4b + | | |
| | | ccr + emd-phaB1 | | |
| Comparative | KNK005 [symbol] phaZ 1, 2, 6 | — | 0.0 | — |
| Example 1 | | | | |
| Comparative | KNK005 [symbol] phaZ 1, 2, 6/nagEG793C, dR | Glucose assimilability | 0.0 | — |
| Example 2 | (Production Example 1) | | | |
| Comparative | pCUP2-lacUV5-cscA in KNK005 [symbol] phaZ 1, 2, 6 | cscA | 0.1 | 0 |
| Example 3 | (Production Example 4) | | | |
| Comparative | pCUP2-lacUV5-cscB in KNK005 [symbol] phaZ 1, 2, 6 | cscB | 0.0 | — |
| Example 4 | (Production Example 5) | | | |

1. A PHA-producing microorganism, comprising:
a PHA synthase gene;
a heterogeneous-organism-derived sucrose hydrolase gene encoding the amino acid sequence of SEQ ID NO: 1, or a variant gene encoding a polypeptide which has a sequence homology of 90% or more to the amino acid sequence of SEQ ID NO: 1 and which has sucrose hydrolase activity; and
a heterogeneous-organism-derived sucrose permease gene encoding the amino acid sequence of SEQ ID NO: 2, or a variant gene encoding a polypeptide which has a sequence homology of 90% or more to the amino acid sequence of SEQ ID NO: 2 and which has sucrose permease activity.

2. The microorganism according to claim 1, which is a transformant in which a microorganism belonging to a genus Cupriavidus is used as a host.

3. The microorganism according to claim 2, wherein the microorganism belonging to the genus Cupriavidus is Cupriavidus necator.

4. The microorganism according to claim 1, wherein glucose assimilability is given to the microorganism, or glucose assimilability is enhanced in the microorganism.

5. The microorganism according to claim 1, wherein the PHA synthase gene is a capable of synthesizing P(3HB-co-3HH).

6. The microorganism according to claim 3, further comprising a crotonyl-CoA reductase gene and an ethylmalonyl-CoA decarboxylase gene.

7. The microorganism according to claim 6, wherein an acetoacetyl CoA reductase gene is deleted, or an expression level thereof is restrained in the microorganism.

8. A method for producing a PHA, comprising:
culturing the microorganism of claim 1 in a medium containing a carbon source comprising sucrose.

9. The method for producing a PHA according to claim 8, wherein the PHA is P(3HB-co-3HH).

10. The microorganism according to claim 1, wherein the variant gene of the sucrose hydrolase gene has a sequence homology of 95% or more to the amino acid sequence of SEQ ID NO: 1 and has sucrose hydrolase activity, and the variant gene of the sucrose permease gene has a sequence homology of 95% or more to the amino acid sequence of SEQ ID NO: 2 and has sucrose permease activity.

11. The microorganism according to claim 1,
wherein the microorganism is a transformant in which a microorganism belonging to a genus Cupriavidus is used as a host, and
glucose assimilability is given to the microorganism, or glucose assimilability is enhanced in the microorganism.

12. The microorganism according to claim 11, wherein the microorganism belonging to the genus Cupriavidus is Cupriavidus necator.

13. The microorganism according to claim 1,
wherein the microorganism is a transformant in which a microorganism belonging to a genus Cupriavidus is used as a host, and
the PHA synthase gene is capable of synthesizing P(3HB-co-3HH).

14. The microorganism according to claim 13, wherein the microorganism belonging to the genus Cupriavidus is Cupriavidus necator.

15. The microorganism according to claim 1,
wherein glucose assimilability is given to the microorganism, or glucose assimilability is enhanced in the microorganism, and
the PHA synthase gene is capable of synthesizing P(3HB-co-3HH).

16. The microorganism according to claim 15, wherein the microorganism is a transformant in which a microorganism belonging to a genus Cupriavidus is used as a host.

17. The microorganism according to claim 16, wherein the microorganism belonging to the genus Cupriavidus is Cupriavidus necator.

18. The microorganism according to claim 1, further comprising a crotonyl-CoA reductase gene and an ethylmalonyl-CoA decarboxylase gene,
wherein glucose assimilability is given to the microorganism, or glucose assimilability is enhanced in the microorganism.

19. The microorganism according to claim 18, wherein the microorganism is a transformant in which a microorganism belonging to a genus Cupriavidus is used as a host.

20. The microorganism according to claim 19, wherein the microorganism belonging to the genus Cupriavidus is Cupriavidus necator.


SEQUENCE LISTING (42 sequences)

SEQ ID NO: 1 - protein, 477 aa, Escherichia coli
MTQSRLHAAQNALAKLHEHRGNTFYPHFHLAPPAGWMNDPNGLIWFNDRYHAFYQHHPMSEHWGPMHWGHATSDDMIHWQHEPIALAPGDDNDKDGCFSGSAVDDNGVLSLIYTGHVWLDGAGNDDAIREVQCLATSRDGIHFEKQGVILTPPEGIMHFRDPKVWREADTWWMVVGAKDPGNTGQILLYRGSSLREWTFDRVLAHADAGESYMWECPDFFSLGDQHYLMFSPQGMNAEGYSYRNRFQSGVIPGMWSPGRLFAQSGHFTELDNGHDFYAPQSFLAKDGRRIVIGWMDMWESPMPSKREGWAGCMTLARELSESNGKLLQRPVHEAESLRQQHQSVSPRTISNKYVLQENAQAVEIQLQWALKNSDAEHYGLQLGTGMRLYIDNQSERLVLWRYYPHENLDGYRSIPLPQRDTLALRIFIDTSSVEVFINDGEAVMSSRIYPQPEERELSLYASHGVAVLQHGALWLLG

SEQ ID NO: 2 - protein, 415 aa, Escherichia coli
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 3 - protein, 589 aa, Cupriavidus necator
MATGKGAAASTQEGKSQPFKVTPGPFDPATWLEWSRQWQGTEGNGHAAASGIPGLDALAGVKIAPAQLGDIQQRYMKDFSALWQAMAEGKAEATGPLHDRRFAGDAWRTNLPYRFAAAFYLLNARALTELADAVEADAKTRQRIRFAISQWVDAMSPANFLATNPEAQRLLIESGGESLRAGVRNMMEDLTRGKISQTDESAFEVGRNVAVTEGAVVFENEYFQLLQYKPLTDKVHARPLLMVPPCINKYYILDLQPESSLVRHVVEQGHTVFLVSWRNPDASMAGSTWDDYIEHAAIRAIEVARDISGQDKINVLGFCVGGTIVSTALAVLAARGEHPAASVTLLTTLLDFADTGILDVFVDEGHVQLREATLGGGAGAPCALLRGLELANTFSFLRPNDLVWNYVVDNYLKGNTPVPFDLLFWNGDATNLPGPWYCWYLRHTYLQNELKVPGKLTVCGVPVDLASIDVPTYIYGSREDHIVPWTAAYASTALLANKLRFVLGASGHIAGVINPPAKNKRSHWTNDALPESPQQWLAGAIEHHGSWWPDWTAWLAGQAGAKRAAPANYGNARYRAIEPAPGRYVKAKA

SEQ ID NO: 4 - protein, 594 aa, Aeromonas caviae
MSQPSYGPLFEALAHYNDKLLAMAKAQTERTAQALLQTNLDDLGQVLEQGSQQPWQLIQAQMNWWQDQLKLMQHTLLKSAGQPSEPVITPERSDRRFKAEAWSEQPIYDYLKQSYLLTARHLLASVDALEGVPQKSRERLRFFTRQYVSAMAPSNFLATNPELLKLTLESGGQNLVRGLALLAEDLERSADQLNIRLTDESAFELGRDLALTPGRVVQRTELYELIQYSPTTETVGKTPVLIVPPFINKYYIMDMRPQNSLVAWLVAQGQTVFMISWRNPGVAQAQIDLDDYVVDGVIAALDGVEAATGEREVHGIGYCIGGTALSLAMGWLAARRQKQRVRTATLFTTLLDFSQPGELGIFIHEPIIAALEAQNEAKGIMDGRQLAVSFSLLRENSLYWNYYIDSYLKGQSPVAFDLLHWNSDSTNVAGKTHNSLLRRLYLENQLVKGELKIRNTRIDLGKVKTPVLLVSAVDDHIALWQGTWQGMKLFGGEQRFLLAESGHIAGIINPPAANKYGFWHNGAEAESPESWLAGATHQGGSWWPEMMGFIQNRDEGSEPVPARVPEEGLAPAPGHYVKVRLNPVFACPTEEDAA

SEQ ID NO: 5 - DNA, 1434 bp, Escherichia coli
atgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaa

SEQ ID NO: 6 - DNA, 1248 bp, Escherichia coli
atggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatag

SEQ ID NO: 7 - DNA, 37 bp, Escherichia coli
tttacactttatgcttccggctcgtataatgtgtgga

SEQ ID NO: 8 - DNA, 32 bp, Artificial
  primer
gcgcatttaaatcctggcgtggttcgtgttcg

SEQ ID NO: 9 - DNA, 20 bp, Artificial
  primer
caaccagcgcgcgccgcgcc

SEQ ID NO: 10 - DNA, 40 bp, Artificial
  primer
ggcgcggcgcgcgctggttggccgcatgctgttctcgatg

SEQ ID NO: 11 - DNA, 32 bp, Artificial
  primer
gcgcatttaaatacagcccgtagcccagcaca

SEQ ID NO: 12 - DNA, 32 bp, Artificial
  primer
gcgcatttaaattcagccattttgcttccaga

SEQ ID NO: 13 - DNA, 20 bp, Artificial
  primer
tgcgaatcctcgtaggtacc

SEQ ID NO: 14 - DNA, 40 bp, Artificial
  primer
ggtacctacgaggattcgcatgcccggcacgcccggcaac

SEQ ID NO: 15 - DNA, 32 bp, Artificial
  primer
gcgcatttaaattggtggtgatgcatggctat

SEQ ID NO: 16 - DNA, 2845 bp, Escherichia coli
ggaaacaattgcacgtgcagagagacaatcaaatcatgacgcaatctcgattgcatgcggcgcaaaacgccctagcaaaacttcatgagcaccggggtaacactttctatccccattttcacctcgcgcctcctgccgggtggatgaacgatccaaacggcctgatctggtttaacgatcgttatcacgcgttttatcaacatcatccgatgagcgaacactgggggccaatgcactggggacatgccaccagcgacgatatgatccactggcagcatgagcctattgcgctagcgccaggagacgataatgacaaagacgggtgtttttcaggtagtgctgtcgatgacaatggtgtcctctcacttatctacaccggacacgtctggctcgatggtgcaggtaatgacgatgcaattcgcgaagtacaatgtctggctaccagtcgggatggtattcatttcgagaaacagggtgtgatcctcactccaccagaaggaatcatgcacttccgcgatcctaaagtgtggcgtgaagccgacacatggtggatggtagtcggggcgaaagatccaggcaacacggggcagatcctgctttatcgcggcagttcgttgcgtgaatggaccttcgatcgcgtactggcccacgctgatgcgggtgaaagctatatgtgggaatgtccggactttttcagccttggcgatcagcattatctgatgttttccccgcagggaatgaatgccgagggatacagttaccgaaatcgctttcaaagtggcgtaatacccggaatgtggtcgccaggacgactttttgcacaatccgggcattttactgaacttgataacgggcatgacttttatgcaccacaaagctttttagcgaaggatggtcggcgtattgttatcggctggatggatatgtgggaatcgccaatgccctcaaaacgtgaaggatgggcaggctgcatgacgctggcgcgcgagctatcagagagcaatggcaaacttctacaacgcccggtacacgaagctgagtcgttacgccagcagcatcaatctgtctctccccgcacaatcagcaataaatatgttttgcaggaaaacgcgcaagcagttgagattcagttgcagtgggcgctgaagaacagtgatgccgaacattacggattacagctcggcactggaatgcggctgtatattgataaccaatctgagcgacttgttttgtggcggtattacccacacgagaatttagacggctaccgtagtattcccctcccgcagcgtgacacgctcgccctaaggatatttatcgatacatcatccgtggaagtatttattaacgacggggaagcggtgatgagtagtcgaatctatccgcagccagaagaacgggaactgtcgctttatgcctcccacggagtggctgtgctgcaacatggagcactctggctactgggttaacgcttgcatgagtgccggcgtgcgtcatgcacggcgccggcaggcctgcaggttccctcccgtttccattgaaaggactacacaatggcactgaatattccattcagaaatgcgtactatcgttttgcatccagttactcatttctcttttttatttcctggtcgctgtggtggtcgttatacgctatttggctgaaaggacatctagggttgacagggacggaattaggtacactttattcggtcaaccagtttaccagcattctatttatgatgttctacggcatcgttcaggataaactcggtctgaagaaaccgctcatctggtgtatgagtttcatcctggtcttgaccggaccgtttatgatttacgtttatgaaccgttactgcaaagcaatttttctgtaggtctaattctgggggcgctattttttggcttggggtatctggcgggatgcggtttgcttgatagcttcaccgaaaaaatggcgcgaaattttcatttcgaatatggaacagcgcgcgcctggggatcttttggctatgctattggcgcgttctttgccggcatattttttagtatcagtccccatatcaacttctggttggtctcgctatttggcgctgtatttatgatgatcaacatgcgttttaaagataaggatcaccagtgcgtagcggcagatgcgggaggggtaaaaaaagaggattttatcgcagttttcaaggatcgaaacttctgggttttcgtcatatttattgtggggacgtggtctttctataacatttttgatcaacaactttttcctgtcttttattcaggtttattcgaatcacacgatgtaggaacgcgcctgtatggttatctcaactcattccaggtggtactcgaagcgctgtgcatggcgattattcctttctttgtgaatcgggtagggccaaaaaatgcattacttatcggagttgtgattatggcgttgcgtatcctttcctgcgcgctgttcgttaacccctggattatttcattagtgaagttgttacatgccattgaggttccactttgtgtcatatccgtcttcaaatacagcgtggcaaactttgataagcgcctgtcgtcgacgatctttctgattggttttcaaattgccagttcgcttgggattgtgctgctttcaacgccgactgggatactctttgaccacgcaggctaccagacagttttcttcgcaatttcgggtattgtctgcctgatgttgctatttggcattttcttcttgagtaaaaaacgcgagcaaatagttatggaaacgcctgtaccttcagcaatatagcctgccggcctggttcaaccagtcggcagccgactagtgcgcgc

SEQ ID NO: 17 - DNA, 62 bp, Artificial
  primer
gcgcgccaattggcgcaacgcaattaatgtgagttagctcactcattaggcaccccaggctt

SEQ ID NO: 18 - DNA, 83 bp, Artificial
  primer
tctctgcacgtgcaattgtttcctgtgtgaaattgttatccgctcacaattccacacattatacgagccggaagcataaagtg

SEQ ID NO: 19 - DNA, 55 bp, Artificial
  primer
ggaaacaattgcacgtgcagagagacaatcaaatcatgacgcaatctcgattgca

SEQ ID NO: 20 - DNA, 64 bp, Artificial
  primer
gcgcgcactagtcggctgccgactggttgaaccaggccggcaggttaacccagtagccagagtg

SEQ ID NO: 21 - DNA, 59 bp, Artificial
  primer
ggaaacaattgcacgtgcagagagacaatcaaatcatggcactgaatattccattcaga

SEQ ID NO: 22 - DNA, 32 bp, Artificial
  primer
gcgcgcactagtcggctgccgactggttgaac

SEQ ID NO: 23 - DNA, 34 bp, Artificial
  primer
gcgcgcatttaaatgcaagcagttcggcgtggcg

SEQ ID NO: 24 - DNA, 25 bp, Artificial
  primer
gcttgctcttcctattcagtcaggg

SEQ ID NO: 25 - DNA, 43 bp, Artificial
  primer
gcagagagacaatcaaatcatgaagacctacgagaacatcgcc

SEQ ID NO: 26 - DNA, 33 bp, Artificial
  primer
gcgcgcatttaaattcagggaaagcgccgcagg

SEQ ID NO: 27 - DNA, 45 bp, Artificial
  primer
ccctgactgaataggaagagcaagctgcttctggcgtcaggcagc

SEQ ID NO: 28 - DNA, 55 bp, Artificial
  primer
gatttgattgtctctctgcacgtgcaattgtttcctgtgtgaaattgttatccgc

SEQ ID NO: 29 - DNA, 32 bp, Artificial
  primer
aaatagatttaaatgggacagcagcaggattt

SEQ ID NO: 30 - DNA, 38 bp, Artificial
  primer
actagtatcgatcaattggccatcagtttatgcctggc

SEQ ID NO: 31 - DNA, 38 bp, Artificial
  primer
caattgatcgatactagtcccggcagccgccagcgcgc

SEQ ID NO: 32 - DNA, 33 bp, Artificial
  primer
aaatagatttaaatacaaaggcaaaggggtagc

SEQ ID NO: 33 - DNA, 2414 bp, Artificial
  artificial synthetic gene
caattgcacgtgcagagagacaatcaaatcatgaaggaaatcctggacgcgattcaggcccagaccgcgaccgcgagcggcaccgccgcggtcacgtccgccgacttcgccgctctccccctgcccgactcgtaccgcgcgatcaccgtgcacaaggacgagacggagatgttcgcgggcctcgagtcccgtgacaaggacccccgcaagtcgctccatctggacgacgtgccgatccccgaactcggccccggtgaggccttggtggccgtcatggcctcctcggtcaactacaactccgtgtggacctcgatcttcgagcccgtctccaccttcagcttcctggagcggtacggccggctcagcgacctgagcaagcgccacgacctgccgtaccacatcatcggctccgacctggcgggcgtcgtgctgcgcaccgggcccggcgtgaacgcctggaacccgggcgacgaggtcgtcgcgcactgcctgagcgtcgagctggagtcctccgacggccacaacgacacgatgctcgaccccgagcagcgcatctggggcttcgagaccaacttcggcggtctcgccgagatcgcgctcgtcaagtccaaccagctcatgccgaagcccggtcacctgagctgggaggaggccgcctcgcccggcctggtgaactccaccgcgtaccgccagctggtgtcccgcaacggcgccggcatgaagcagggcgacaacgtgctgatctggggcgcgagcggcggactcgggtcgtacgccacgcagttcgcgctcgccggcggcgccaaccccatctgtgtcgtctccagcccccagaaggcggagatctgccgcgcgatgggcgccgaggcgatcatcgaccgcaacgccgagggctacaagttctggaaggacgagcagacccaggaccccaaggagtggaagcgcttcggcaagcgcatccgcgagctcaccggcggcgaggacatcgacatcgtcttcgagcaccccggccgcgagaccttcggcgcctcggtctacgtcacgcgcaagggcggcaccatcaccacctgcgcctcgacctcgggctacatgcacgagtacgacaaccgctacctgtggatgtccctgaagcgcatcatcggctcgcacttcgccaactaccgcgaggcgtgggaggccaaccgcctgatcgccaagggcaagatccacccgacgctctccaagacgtaccgcctggaggacaccggccaggccgcctacgacgtccaccgcaacctccaccagggcaaggtcggcgtcctcgccctcgcgcccgaggagggcctgggcgtgcgcgacccggagaagcgggcccagcacatcgacgcgatcaaccgtttccgcaacgtctgacgcttgcatgagtgccggcgtgcgtcatgcacggcgccggcaggcctgcaggttccctcccgtttccattgaaaggactacacaatggccaagtgcctgctgacgtccagcctgagcgtgcggaccaagctgctccagaccggcgtgtcgctgtacaacacgtcgcatgggttccacgaggaggaggtcaagaagatcctggagcagtttccgggcggctcgatcgacctgctgaagaagcagaacggcatcggcatcctcacgctgaacaacccgaacaagatgaacgcgttctccggggtgatgatgctccagctgctggagcgcgtgatcgaactggagaactggaccgaaggcaaaggcctcatcatccatggcgcgaagaacaccttctgctcggggtcggacctgaatgccgtgaaagccctgagcacgccggaatcgggcgtggcgctgagcatgttcatgcagaacaccctgacccgcttcatgcggctgccgctgatctcggtcgcactggtgcaaggctgggccatgggcggcggcgccgaactgacgaccgcgtgcgactttcgcctgatgacggaggagtcggtcattcgcttcgtgcacaaggagatgggcatcgtcccgtcgtggggcggcacctcccgcctggtggagatcatcggcagccgccaagcgctgaaggtcctgagcggcaccctgaagctggactcgaaggaggccctgaacatcggcctgaccgatgaggtgctccagccgtcggacgaaaccaccgccctggaacaggcccaggaatggctggagaagttcgtgagcggcccgccccaggtcattcgcggcctgaagaagtccgtgtgcagcgcacgcgagctgtatatcgaggaggcgctccagaatgaacgcgatgtgctggaaacgctgtggggcggccccgccaatctggaagccatcgcgaagaagggcaagcacaccaagtagcctgccggcctggttcaaccagtcggcagccgactagt

SEQ ID NO: 34 - DNA, 29 bp, Artificial
  primer
gcgcgccaattgtgcttctggcgtcaggc

SEQ ID NO: 35 - DNA, 35 bp, Artificial
  primer
gcgcgccaattgtttcctgtgtgaaattgttatcc

SEQ ID NO: 36 - DNA, 34 bp, Artificial
  primer
gcgcgcatttaaatcacctggcagggcatgaagc

SEQ ID NO: 37 - DNA, 30 bp, Artificial
  primer
tgtgtagtcctttcaatggaaacgggaggg

SEQ ID NO: 38 - DNA, 43 bp, Artificial
  primer
cgtttccattgaaaggactacacacctgccggcctggttcaac

SEQ ID NO: 39 - DNA, 34 bp, Artificial
  primer
gcgcgcatttaaatctgtccttgtgtcatcgctg

SEQ ID NO: 40 - protein, 453 aa, Streptomyces cinnamonensis
MKEILDAIQAQTATASGTAAVTSADFAALPLPDSYRAITVHKDETEMFAGLESRDKDPRKSLHLDDVPIPELGPGEALVAVMASSVNYNSVWTSIFEPVSTFSFLERYGRLSDLSKRHDLPYHIIGSDLAGVVLRTGPGVNAWNPGDEVVAHCLSVELESSDGHNDTMLDPEQRIWGFETNFGGLAEIALVKSNQLMPKPGHLSWEEAASPGLVNSTAYRQLVSRNGAGMKQGDNVLIWGASGGLGSYATQFALAGGANPICVVSSPQKAEICRAMGAEAIIDRNAEGYKFWKDEQTQDPKEWKRFGKRIRELTGGEDIDIVFEHPGRETFGASVYVTRKGGTITTCASTSGYMHEYDNRYLWMSLKRIIGSHFANYREAWEANRLIAKGKIHPTLSKTYRLEDTGQAAYDVHRNLHQGKVGVLALAPEEGLGVRDPEKRAQHIDAINRFRNV

SEQ ID NO: 41 - protein, 299 aa, Mus musculus
MAKCLLTSSLSVRTKLLQTGVSLYNTSHGFHEEEVKKILEQFPGGSIDLLKKQNGIGILTLNNPNKMNAFSGVMMLQLLERVIELENWTEGKGLIIHGAKNTFCSGSDLNAVKALSTPESGVALSMFMQNTLTRFMRLPLISVALVQGWAMGGGAELTTACDFRLMTEESVIRFVHKEMGIVPSWGGTSRLVEIIGSRQALKVLSGTLKLDSKEALNIGLTDEVLQPSDETTALEQAQEWLEKFVSGPPQVIRGLKKSVCSARELYIEEALQNERDVLETLWGGPANLEAIAKKGKHTK

SEQ ID NO: 42 - DNA, 900 bp, Artificial
  artificial synthetic gene
atggccaagtgcctgctgacgtccagcctgagcgtgcggaccaagctgctccagaccggcgtgtcgctgtacaacacgtcgcatgggttccacgaggaggaggtcaagaagatcctggagcagtttccgggcggctcgatcgacctgctgaagaagcagaacggcatcggcatcctcacgctgaacaacccgaacaagatgaacgcgttctccggggtgatgatgctccagctgctggagcgcgtgatcgaactggagaactggaccgaaggcaaaggcctcatcatccatggcgcgaagaacaccttctgctcggggtcggacctgaatgccgtgaaagccctgagcacgccggaatcgggcgtggcgctgagcatgttcatgcagaacaccctgacccgcttcatgcggctgccgctgatctcggtcgcactggtgcaaggctgggccatgggcggcggcgccgaactgacgaccgcgtgcgactttcgcctgatgacggaggagtcggtcattcgcttcgtgcacaaggagatgggcatcgtcccgtcgtggggcggcacctcccgcctggtggagatcatcggcagccgccaagcgctgaaggtcctgagcggcaccctgaagctggactcgaaggaggccctgaacatcggcctgaccgatgaggtgctccagccgtcggacgaaaccaccgccctggaacaggcccaggaatggctggagaagttcgtgagcggcccgccccaggtcattcgcggcctgaagaagtccgtgtgcagcgcacgcgagctgtatatcgaggaggcgctccagaatgaacgcgatgtgctggaaacgctgtggggcggccccgccaatctggaagccatcgcgaagaagggcaagcacaccaagtag